(19) United States
(12) Patent Application Publication
(10) Pub. No.: US 2024/0156970 A1
(43) Pub. Date: May 16, 2024

(54) PATHOGEN MOIETIES AND USES THEREOF

(71) Applicants: GRIFFITH UNIVERSITY, Nathan (AU); University of Iowa Research Foundation, Iowa City, IA (US)

(72) Inventors: Michael Jennings, Nathan (AU); Feda (En-Chi) Jen, Nathan (AU); Kate Seib, Nathan (AU); Evegny Semchenko, Nathan (AU); Milton Kiefel, Nathan (AU); Michael Apicella, Iowa City, IA (US)

(21) Appl. No.: 18/550,659

(22) PCT Filed: Mar. 14, 2022
(86) PCT No.: PCT/AU2022/050213

§ 371 Date: Sep. 14, 2023

Related U.S. Application Data
(60) Provisional application No. 63/161,399, filed on Mar. 15, 2021.

(51) Int. Cl. A61K 47/62; A61K 39/00; A61K 39/095

(52) U.S. Cl. CPC A61K 47/62; A61K 39/095; A61K 2039/6037; A61K 2039/6081

(57) ABSTRACT

This disclosure relates generally to Neisseria surface moieties. More particularly, the present disclosure relates to oligosaccharides corresponding to Neisseria lipooligosaccharides and to chimeric molecules comprising these moieties for eliciting an immune response to Neisseria organisms and/or for treating or inhibiting the development of Neisseria infections.

### RELATED APPLICATIONS

This application claims priority to U.S. Provisional Application No. 63/161,399 entitled “Pathogen moieties and uses thereof” filed 15 Mar. 2021, the contents of which are incorporated herein by reference in their entirety.

### GOVERNMENT FUNDING

This invention was made with Government support under Grant Nos. NIAD AI108255 and NAID AI024616 awarded by the National Institute of Allergy and Infectious Diseases. The Government has certain rights in the invention.

### FIELD

This disclosure relates generally to Neisseria surface moieties. More particularly, the present disclosure relates to oligosaccharides corresponding to Neisseria lipooligosaccharides and to chimeric molecules comprising these moieties for eliciting an immune response to Neisseria organisms and/or for treating or inhibiting the development of Neisseria infections.

### BACKGROUND

Neisseria gonorrhoeae is a host adapted bacterial pathogen that causes the sexually transmitted disease gonorrhea. Gonococcal infections can be symptomatic or asymptomatic, with up to 80% of infections in females being asymptomatic. Untreated gonorrhea in females can lead to pelvic inflammatory disease, adverse pregnancy outcomes, neonatal complications, or infertility. Moreover, it can increase the risk of acquiring and transmitting HIV. It is estimated that there are more than 106 million cases of gonorrhea worldwide each year and the emergence of multidrug resistant (MDR) strains of N. gonorrhoeae is a major public health problem. Isolates with resistance to nearly every antibiotic used to treat gonorrhea have been identified. In the past century, only three potential gonococcal vaccines have been tested in clinical trials and none of these vaccines was successful. As such, there is an urgent unmet need for novel approaches for therapeutic and vaccine development.

Lipooligosaccharide (LOS) is one of the major structural components of the outer membrane and is a key virulence factor of N. gonorrhoeae. It plays a number of key roles in the pathogenesis of N. gonorrhoeae, including mediating direct interaction between N. gonorrhoeae and human urethral epithelial cells (Harvey et al. 2000, Mol Microbiol. 36:1059-70 and Song et al. 2000, J Exp Med 191:949-60). The lipid A of LOS of N. gonorrhoeae binds to complement component C3 which is required for activation of complement receptor 3 in primary cervical epithelial cells (Edwards et al. 2002, Cell Microbiol 4:585-98). Bacteria, such as N. gonorrhoeae, can mimic host structures to avoid detection. An aspect of this mimicry is the expression of sialic acid, a sugar structure found in humans, on the bacterial surface. The LOS structure of N. gonorrhoeae can be terminated with a N-acetylneuraminic acid (Neu5Ac), which has multiple roles in gonococcal virulence (Apicella et al. 1989, Pediatr Infect Dis J. 8:901-2 and Mandrell et al. 1993, Immunobiology 187:382-402). However, N. gonorrhoeae cannot synthesize the nucleotide sugar precursor, cytidine-5′-monophosphate (CMP)-Neu5Ac that is required for LOS biosynthesis and must acquire CMP-Neu5Ac from the host (Mandrell et al. 1993, Immunobiology 187:382-402). In contrast, the closely related pathogen Neisseria meningitidis can synthesize CMP-Neu5Ac, which is also required for the polysaccharide capsule structure of meningococcal serogroup B, C, W and Y. Both of these pathogenic Neisseria species have an almost identical (95% amino acid identity) sialyltransferase, Lst, that transfers Neu5Ac from CMP-Neu5Ac to the terminal galactose of LOS as shown in FIG. 1 (Gilbert et al. 1996, J Biol Chem 271:28271-6). The Lst sialyltransferase expressed by the pathogenic Neisseria species is homologous to the LsgB sialyltransferase of non-typeable Haemophilus influenzae (NTHi).

### SUMMARY

The present disclosure is predicated in part on the finding that a bacterial-specific sugar, keto-deoxyoctulosonate (KDO), is commonly found as the terminal LOS sugar instead of sialic acid on N. gonorrhoeae and N. meningitidis and that KDO is added by the sialyltransferase Lst as shown in FIG. 1. This finding is entirely unexpected, as in both organisms Lst, the enzyme that adds sialic acid to LOS was previously considered to be on the outside of the bacterial cell (Shell et al. 2002, Infect Immun. 70:3744-51), whereas the donor molecule for adding KDO to biomolecules, cytidine-monophosphate KDO (CMP-KDP), was considered to be in the cytoplasm of the bacterial cell. The present finding represents an important paradigm shift in terms of vaccine development, as this knowledge allows the bacterial KDO carbohydrate to be considered as a target for the development of new strategies to prevent and treat Neisseria infections including N. gonorrhoeae and N. meningitidis infections. It is proposed that a KDO-based vaccine will elicit an immune response that is distinct to that elicited during natural infections and that the antibodies targeting this bacterial KDO epitope will mediate bactericidal and opsonophagocytic killing, thus providing immune protection against Neisseria infections.

Accordingly, disclosed herein in one aspect is a chimeric molecule comprising, consisting or consisting essentially of a carrier and an oligosaccharide comprising an oligosaccharide sequence corresponding to a Neisseria lipooligosaccharide (LOS) (e.g., a N. gonorrhoeae or N. meningitidis LOS), wherein the oligosaccharide sequence comprises keto-deoxyoctulosonate (KDO) as a terminal saccharide unit. The oligosaccharide sequence suitably comprises the KDO attached to galactose (Gal), typically wherein the Gal is further attached to N-acetylglucosamine (GlcNAc). In representative embodiments, the KDO is attached to Gal via an α(2→3) linkage and/or the Gal is attached to GlcNAc via a β(1→4) linkage. In illustrative examples of this type, the oligosaccharide sequence comprises KDOα(2→3)Gal, or KDOα(2→3)Galβ(1→4)GlcNAc.

In some embodiments, the oligosaccharide comprises n occurrences of the oligosaccharide sequence, wherein n is 1 to 4. In representative examples of this type, n is 2. Suitably, respective oligosaccharide sequences are connected to one another by a linker.

The carrier is typically connected to the oligosaccharide by a linker. Suitably, the carrier is a protein, a peptide, a lipid, a polymer, a dendrimer, a virosome, a virus-like particle (VLP), an outer membrane vesicle or a combination thereof. In specific embodiments, the carrier is a protein carrier, which can be for example a bacterial toxoid, a toxin, an exotoxin, and a nontoxic derivative thereof, such as keyhole limpet hemocyanine (KLH), hepatitis B virus core protein, thyroglobulin, albumins (such as bovine serum albumin (BSA), human serum albumin (HSA), and ovalbumin), pneumococcal surface protein A (PspA), pneumococcal adhesin protein (PsaA), purified protein derivative of tuberculin (PPD); transferrin binding proteins, polyamino acids, such as poly(lysine:glutamic acid), tetanus toxoid, tetanus toxin Fragment C, diphtheria toxoid, CRM (a nontoxic diphtheria toxin mutant), cholera toxoid, Staphylococcus aureus exotoxins or toxoids, Escherichia coli heat labile enterotoxin, Pseudomonas aeruginosa exotoxin A and bacterial outer membrane proteins (such as N. meningitidis serotype B outer membrane protein complex (OMPC) and outer membrane class 3 porin (rPorB)). In specific embodiments, the protein carrier is CRM197.

Another aspect of the present disclosure relates to a composition comprising, consisting or consisting essentially of a chimeric molecule as broadly described above and elsewhere herein, and a pharmaceutically acceptable vehicle or adjuvant. The composition may optionally comprise one or more ancillary agents for treating or inhibiting the development of a Neisseria infection. The ancillary agents may be selected from antimicrobial agents and immunogens (e.g., proteinaceous immunogens derived from Neisseria antigens such as AniA, MsrAB, MetQ, NhbA, etc.) for eliciting an immune response to a Neisseria organism.

Disclosed herein in another aspect is a method of eliciting an immune response to a Neisseria organism in a subject, the method comprising, consisting or consisting essentially of immunizing the subject with a chimeric molecule or composition as broadly described above and elsewhere herein, to thereby elicit an immune response to the Neisseria organism in the subject. Preferably, the Neisseria organism is N. gonorrhoeae or N. meningitidis. In some embodiments, the Neisseria organism is resistant to at least one antibacterial drug.

Still another aspect of the present disclosure relates to a method of producing an antigen-binding molecule that binds specifically with a Neisseria organism, the method comprising: (1) immunizing an animal with a chimeric molecule or composition as broadly described above and elsewhere herein; (2) detecting a B cell from the animal, which binds specifically with the chimeric molecule (particularly its oligosaccharide component) or the LOS of the Neisseria organism; and (3) isolating the antigen-binding molecule expressed by that B cell.

Also disclosed herein in another aspect is a method of producing an antigen-binding molecule that binds specifically with a Neisseria organism, the method comprising: (1) screening a library of antigen-binding molecules with a chimeric molecule or composition as broadly described above and elsewhere herein; (2) detecting an antigen-binding molecule that binds specifically with the chimeric molecule (particularly its oligosaccharide component) or the LOS of the Neisseria organism; and (3) isolating the detected antigen-binding molecule.

In a related aspect, the present disclosure relates to an antigen-binding molecule produced by the methods broadly described above and elsewhere herein, or a derivative antigen-binding molecule with the same epitope-binding specificity as the antigen-binding molecule. In representative embodiments, the derivative antigen-binding molecule is selected from antibody fragments (such as Fab, Fab′, F(ab′)₂, Fv), single chain (scFv) and domain antibodies (including, for example, shark and camelid antibodies), and fusion proteins comprising an antibody, and any other modified configuration of the immunoglobulin molecule that comprises an antigen binding/recognition site.

In another related aspect, the present disclosure relates to a cell (e.g., a hybridoma or cell line) that produces the antigen-binding molecule as broadly described above and elsewhere herein.

Also disclosed herein is a method for treating or inhibiting the development of a Neisseria infection in a subject, the method comprising administering to the subject an effective amount of a chimeric molecule, composition or antigen-binding molecule as broadly described above and elsewhere herein. In some embodiments, the chimeric molecule or antigen-binding molecule is administered concurrently with an ancillary agent (e.g., an antimicrobial agent and/or immunogen).

Disclosed herein in yet another aspect is a method for detecting the presence of a Neisseria organism in a subject, the method comprising contacting a biological sample taken from the subject with an antigen-binding molecule that binds specifically with a Neisseria organism as broadly described above and elsewhere herein and detecting the presence of a complex comprising the Neisseria organism or part thereof (e.g., LOS) to thereby detect the presence of the Neisseria organism in the subject.

Yet another aspect of the present disclosure relates to a kit for treating or inhibiting the development of a Neisseria infection in a subject, the kit comprising: a chimeric molecule, composition, or antigen-binding molecule as broadly described above and elsewhere herein, and optionally instructional material for performing the treatment or inhibition.

In still another aspect, a kit is provided for detecting the presence of a Neisseria organism in a subject, the kit comprising: an antigen-binding molecule as broadly described above and elsewhere herein, and optionally instructional material for performing the detection.

### BRIEF DESCRIPTION OF THE DRAWINGS

FIG. 1 is an illustration depicting the structure of the KDO-containing LOS of N. gonorrhoeae strain 1291. MAb 6E4 only recognizes the terminal KDO glycan. Lst is the sialyltransferase which can also incorporate KDO onto the terminal Gal, in the absence of Neu5Ac. LgtB is the galactosyltransferase that transfers the KDO acceptor galactose (Gal) to N-acetylglucosamine (GlcNAc). Without Lst, Gal is the terminal sugar of LOS in N. gonorrhoeae.

FIG. 2 is a graphical and photographical representation showing whole-cell ELISA of 6E4. (A) ELISA of whole-cell N. gonorrhoeae WT and lst mutant strains using 6E4 MAb. Lst transfers the terminal sialic acid to the terminal Gal of LOS in N. meningitidis. Here, the lst mutant showed Lst also transfers KDO to the terminal Gal in N. gonorrhoeae. (B) Whole-cell ELISA of 6E4 was performed to test 20 clinical N. gonorrhoeae isolates. 1291 and 1291lst were the positive and negative controls in this ELISA. (C) ELISA of whole-cell N. meningitidis MC58 WT, siaB, and lst mutant strains using 6E4 MAb. MC58 WT has Neu5Ac as the terminal glycan of LOS. Without SiaB, N. meningitidis cannot make CMP-Neu5Ac; therefore, Lst adds KDO at the terminal of LOS instead of sialic acid. (D) Incorporation of KDO on gonococcal LOS. Whole-cell ELISA of 6E4 showed that the presence of CMP-Neu5Ac in the growing medium could inhibit the incorporation of KDO into the terminal Gal. N. gonorrhoeae 1291 and lst mutant were grown in 0, 30, and 100 μg/mL CMP-Neu5Ac supplemented media. (E) Silver staining and 6E4 Western blot of purified LOS samples from N. meningitidis MC58, N. gonorrhoeae 1291 wild-type and glycosyltransferase mutant strains.

FIG. 3 is a photographic representation depicting a confocal microscopy study of cervical swab from a patient with a documented N. gonorrhoeae infection stained with both anti-KDO MAb 6E4 (Texas red) and anti-LOS MAb 664 (FITC). Infected polymorphonuclear leukocytes (PMNs) can be seen with gonococci stained with both antibodies separately (red or green) and with antibodies colocalized to the same organisms (yellow). This study indicates that KDO termination occurs in vivo as well as in vitro.

FIG. 4 is a graphical representation showing the functional activity of Mab 6E4 against N. gonorrhoeae. The survival of the N. gonorrhoeae in the presence of 0-10 μg/mL Mab 6E4, primary human PMNs and 10% normal human serum as a complement source is shown. Data represent the average survival (+1 standard deviation) for triplicate samples, shown as a percentage of bacteria in the absence of antibody (the 0 μg/mL Mab 6E4 set at 100%, represents 5.07×10³ CFU). There was a statistically significant difference between groups as determined by one-way ANOVA (F(6,14)=59.20, P=<0.0001). Statistically significant differences relative to the untreated wild type, using a two-tailed Student's t test are indicated: * P<0.05; ** P≤0.01; *** P≤0.001. (P-values: 1.25 μg/mL, 0.012; 2.5 μg/mL, 0.005; 5 μg/mL, 0.001; 10 μg/mL, 0.0003).

FIG. 5 is a schematic representation showing chemical synthesis of KDO-spacer. A protected form of KDO, synthesized using published chemistry, is converted into the glycosyl fluoride derivative which is then reacted with the spacer unit using standard Lewis Acid catalyzed glycosidation chemistry. All protecting groups that have been installed to facilitate the chemical synthesis are then removed to give the final compound ready for conjugation.

FIG. 6 is an illustration and photographic representation depicting (A) Coupling of NH₂-linked KDO to CRM197. (13) After conjugation, CRM197 (Coomassie, lane 2) shifts to a higher apparent molecular weight (Coomassie, lane 1) and becomes KDO positive (Western blot with 6E4, lane 1) relative to unconjugated CRM197.

FIG. 7 is a schematic representation showing chemical synthesis of KDOα(2,3)Gal-spacer. A protected form of KDO, synthesized using published chemistry, is converted into the glycosyl fluoride derivative. Commercially available galactose is converted into the Gal-spacer derivative using standard carbohydrate chemistry manipulations. The KDO fluoride and the Gal-spacer unit are combined together using standard Lewis Acid catalyzed glycosidation chemistry. All protecting groups that have been installed to facilitate the chemical synthesis are then removed using standard methods to give the final compound ready for conjugation.

FIG. 8 is a schematic representation showing chemical synthesis of KDOα(2,3)Galβ(1,4)GlcNAc-spacer. A protected form of KDO, synthesized using published chemistry, is converted into the glycosyl fluoride derivative. Commercially available lactulose is converted into the Gal-GlcNAc-spacer derivative using standard carbohydrate chemistry manipulations. The KDO fluoride and the Gal-GlcNAc-spacer unit are combined together using standard Lewis Acid catalyzed glycosidation chemistry. All protecting groups that have been installed to facilitate the chemical synthesis are then removed using standard methods to give the final compound ready for conjugation.

FIG. 9 is a schematic representation showing a chemoenzymic approach to synthesize KDO (A) di- and (B) tri-saccharide antigens. KdsB and Lst in green are enzymes involved in synthesis.

FIG. 10 is a schematic and graphical representation showing (A) in vivo biosynthetic pathway of LOS containing the KDO antigen in outer membrane vesicles (OMVs; the inset electron microscopy image, which shows OMV production typical of E. coli strains with a dual mutations lacking expression of OmpA and Braun's lipoprotein, is modified from Sonntag et al. (1978, J. Bacteriol. 136:280-285). Lacto-N-neotetraose (LNnT) is synthesized by LgtE, LgtA and LgtB. Up-regulation of Lst and KdsB (in orange) will increase the modification of terminal KDO to the LNnT. The synthesized LOS will be transported to the outer membrane. (B) LgtE, LgtA, LgtB and Lst can be co-expressed and produce the same terminal KDO glycan as Ng in E. coli. The terminal KDO is confirmed by ELISA using mAb 6E4. * P<0.05.

### DETAILED DESCRIPTION

### 1. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

The term “adjuvant” as used herein refers to a compound that, when used in combination with a specific immunogen (e.g., a chimeric molecule of the present disclosure) in a composition, will augment the resultant immune response, including intensification or broadening the specificity of either or both antibody and cellular immune responses. In the context of the present disclosure, an adjuvant will preferably enhance the specific immunogenic effect of the active agents of the present disclosure. The term “adjuvant” is typically understood not to comprise agents which confer immunity by themselves. An adjuvant assists the immune system unspecifically to enhance the antigen-specific immune response by e.g., promoting presentation of an antigen to the immune system or induction of an unspecific innate immune response. Furthermore, an adjuvant may preferably e.g., modulate the antigen-specific immune response by e.g., shifting the dominating Th2-based antigen specific response to a more Th1-based antigen specific response or vice versa. Accordingly, an adjuvant may favorably modulate cytokine expression/secretion, antigen presentation, type of immune response etc.

The terms “administration concurrently” or “administering concurrently” or “co-administering” and the like refer to the administration of a single composition containing two or more agents, or the administration of each agent as separate compositions and/or delivered by separate routes either contemporaneously or simultaneously or sequentially within a short enough period of time that the effective result is equivalent to that obtained when all such agents are administered as a single composition. By “simultaneously” is meant that the agents are administered at substantially the same time, and desirably together in the same composition. By “contemporaneously” it is meant that the agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The agents may be administered in either order. The term “sequentially” as used herein means that the agents are administered in sequence, for example at an interval or intervals of minutes, hours, days or weeks. If appropriate the agents may be administered in a regular repeating cycle.

As used herein, “and/or” refers to and encompasses any and all possible combinations of one or more of the associated listed items, as well as the lack of combinations when interpreted in the alternative (or).

As used herein, the term “antigen” and its grammatically equivalents expressions (e.g., “antigenic”) refer to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present disclosure include polyclonal and monoclonal antibodies as well as their fragments (such as Fab, Fab′, F(ab′)₂, Fv), single chain (scFv) and domain antibodies (including, for example, shark and camelid antibodies), and fusion proteins comprising an antibody, and any other modified configuration of the immunoglobulin molecule that comprises an antigen binding/recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known. Antigen-binding molecules also encompass dimeric antibodies, as well as multivalent forms of antibodies. In some embodiments, the antigen-binding molecules are chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, for example, U.S. Pat. No. 4,816,567; and Morrison et al., 1984, Proc. Natl. Acad. Sci. USA 81:6851-6855). Also contemplated, are humanized antibodies, which are generally produced by transferring complementarity determining regions (CDRs) from heavy and light variable chains of a non-human (e.g., rodent, preferably mouse) immunoglobulin into a human variable domain. Typical residues of human antibodies are then substituted in the framework regions of the non-human counterparts. The use of antibody components derived from humanized antibodies obviates potential problems associated with the immunogenicity of non-human constant regions. General techniques for cloning non-human, particularly murine, immunoglobulin variable domains are described, for example, by Orlandi et al. (1989, Proc. Natl. Acad. Sci. USA 86: 3833). Techniques for producing humanized monoclonal antibodies are described, for example, by Jones et al. (1986, Nature 321:522), Carter et al. (1992, Proc. Natl. Acad. Sci. USA 89: 4285), Sandhu (1992, Crit. Rev. Biotech. 12: 437), Singer et al. (1993, J. Immun. 150: 2844), Sudhir (ed., Antibody Engineering Protocols, Humana Press, Inc. 1995), Kelley (“Engineering Therapeutic Antibodies,” in Protein Engineering: Principles and Practice Cleland et al. (eds.), pages 399-434 (John Wiley & Sons, Inc. 1996), and by Queen et al., U.S. Pat. No. 5,693,762 (1997). Humanized antibodies include “primatized” antibodies in which the antigen-binding region of the antibody is derived from an antibody produced by immunizing macaque monkeys with the antigen of interest. Also contemplated as antigen-binding molecules are humanized antibodies.

The term “antimicrobial agent” as used herein refers to any agent with antimicrobial activity, i.e., the ability to inhibit or reduce the growth and/or kill a microbe, e.g., by at least about 30%, at least about 40%, at least about 50%, at least about 75%, at least about 90% or more, as compared to in the absence of an antimicrobial agent. The term “antimicrobial agent” encompasses agents that that inhibit or reduce the growth and/or kill a microbe by directly interacting with the microbe and/or cells of the host in which the microbe resides or is located. Non-limiting examples of antimicrobial agents include a silver nanoparticle, a small molecule, a peptide, a peptidomimetics, an antibody or a fragment thereof, a nucleic acid, an enzyme (e.g., an antimicrobial metalloendopeptidase such as lysostaphin), an aptamer, a drug, an antibiotic, a chemical or any entity that can inhibit the growth and/or kill a microbe. Examples of an antimicrobial peptide that can be included in the compositions described herein, include, but are not limited to, mefloquine, venturicidin A, antimycin, myxothiazol, stigmatellin, diuron, iodoacetamide, potassium tellurite hydrate, aDL-vinylglycine, N-ethylmaleimide, L-allyglycine, diaryquinoline, betaine aldehyde chloride, acivcin, psicofuraine, buthionine sulfoximine, diaminopemelic acid, 4-phospho-D-erythronhydroxamic acid, motexafin gadolinium and/or xycitrin or modified versions or analogues thereof. Representative antimicrobial agents include, antibiotics, antifungals, antiprotozoals, antimalarials, antituberculotics and antivirals, and any mixtures thereof.

As used herein, the term “binds specifically” refers to a binding reaction which is determinative of the presence of a chimeric polypeptide or complex of the present disclosure in the presence of a heterogeneous population of molecules including macromolecules such as proteins and other biologics. In specific embodiments, the term “binds specifically” when referring to an antigen-binding molecule is used interchangeably with the term “specifically immuno-interactive” and the like to refer to a binding reaction which is determinative of the presence of a chimeric polypeptide or complex of the present disclosure in the presence of a heterogeneous population of proteins and other biologics. Under designated assay conditions, a molecule binds specifically to a chimeric polypeptide or complex of the disclosure and does not bind in a significant amount to other molecules (e.g., proteins or antigens) present in the sample. In antigen-binding molecule embodiments, a variety of immunoassay formats may be used to select antigen-binding molecules that are specifically immuno-interactive with a chimeric polypeptide or complex of the disclosure. For example, solid-phase ELISA immunoassays are routinely used to select monoclonal antibodies that are specifically immuno-interactive with a protein. See Harlow and Lane (1988) Antibodies, A Laboratory Manual, Cold Spring Harbor Publications, New York, for a description of immunoassay formats and conditions that can be used to determine specific immunoreactivity.

As used herein, the term “carrier” refers to a protein, a peptide, a lipid, a polymer, a dendrimer, a virosome, a virus-like particle (VLP), an outer membrane vesicle or a combination thereof, which is coupled to an oligosaccharide of the present disclosure to enhance the immunogenicity of the resulting oligosaccharide-carrier chimeric molecule to a greater degree than the oligosaccharide alone. Likewise, the term “protein carrier”, as used herein, refers to a protein or peptide, which is operably linked to an oligosaccharide to enhance the immunogenicity of the resulting oligosaccharide-protein carrier chimeric molecule to a greater degree than the oligosaccharide alone. For example, when used as a carrier, the protein carrier may serve as a T-dependent antigen which can activate and recruit T-cells and thereby augment T-cell dependent antibody production.

The term “chimeric”, when used in reference to a molecule, means that the molecule contains portions that are derived from, obtained or isolated from, or based upon two or more different origins or sources. In specific embodiments, a chimeric molecule of the present disclosure is an immunogenic chimeric molecule for stimulating or eliciting a specific immune response in an animal. In some embodiments, the immune response is protective in that it enables the animal to better resist infection from the organism against which the immunogenic chimeric molecule is directed.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the terms “conjugated”, “linked”, “fused” or “fusion” and their grammatical equivalents, in the context of joining together of two more elements or components or domains by whatever means including chemical conjugation or recombinant means are used interchangeably. Methods of chemical conjugation (e.g., using heterobifunctional crosslinking agents) are known in the art and includes covalent and/or non-covalent linkages. As used herein, “conjugate” refers to an oligosaccharide chemically coupled to a carrier through a linker and/or a cross-linking agent.

By “effective amount”, in the context of treating, inhibiting the development of, or preventing a condition is meant the administration of an amount of an agent or composition to an individual in need of such treatment, inhibition or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials. For example, non-limiting symptoms of N. gonorrhoeae infections include pain in the lower abdomen, pelvis, testicle, or vagina, painful urination, abnormal vaginal discharge, discharge from penis, or increased vaginal discharge, fever, frequent urge to urinate, irregular menstruation, pus, or sore throat. Representative examples of N. meningitidis infections include fever and chills, fatigue, vomiting, cold hands and feet, severe aches or pains in the muscles, joints, chest, or abdomen, rapid breathing diarrhea, and in later stages, dark purple rash.

The term “epitope” means an antigenic determinant that is specifically bound by an antigen-binding molecule or an antigen receptor such as a B-cell receptor (BCR) or T-cell receptor (TCR). Epitopes usually consist of surface groupings of molecules, such as amino acids and/or sugar side chains, and may be linear or have specific three-dimensional structural characteristics, as well as specific charge characteristics. B cell epitope sites on proteins, oligosaccharides, or other biopolymers may be composed of moieties from different parts of the macromolecule that have been brought together by folding. Epitopes of this kind are referred to as conformational or discontinuous epitopes, since the site is composed of segments of the polymer that are discontinuous in the linear sequence but are continuous in the folded conformation(s). Epitopes that are composed of single segments of biopolymers or other molecules are termed continuous or linear epitopes. T cell epitopes are generally restricted to linear peptides. Antigen-binding molecules that recognize the same epitope can be identified in a simple immunoassay showing the ability of one antigen-binding molecule to block the binding of another antigen-binding molecule to a target antigen.

As used herein, the terms “inhibit”, “inhibits”, or “inhibition” (and grammatical equivalents thereof) are not meant to imply complete abolition of disease and encompasses any type of prophylactic treatment that reduces the incidence of the condition, delays the onset of the condition, and/or reduces the symptoms associated with the condition after onset.

An “immune response” as used herein, refers to a response by the immune system of a subject. For example, an immune response may be to an antigen/immunogen that the subject's immune system recognizes as foreign (e.g., non-self-antigens) or self (e.g., self-antigens recognized as foreign). Immune responses may be humoral, involving production of immunoglobulins or antibodies, or cellular, involving various types of B and T lymphocytes, dendritic cells, macrophages, antigen presenting cells and the like, or both. Immune responses may also involve the production or elaboration of various effector molecules such as cytokines. The term “immune response” encompasses immunogenic responses that cause, activate, elicit, stimulate, or induce an immune response against a particular antigen (e.g., an antigen of a pathogenic organism) or organism (e.g., a pathogenic microorganism) in a subject, as well as immunosuppressive or tolerogenic immune responses that inhibit, suppress, diminish or eliminate an immune response, or render the immune system unresponsive, or delay the occurrence or onset of an immune response, to an allergen, or to a self-antigen or a cell, tissue or organ that expresses such an antigen.

Reference herein to “immuno-interactive” includes reference to any interaction, reaction, or other form of association between molecules and in particular where one of the molecules is, or mimics, a component of the immune system.

As used herein, the term “immunity” refers to protection from disease (e.g., preventing or attenuating (e.g., suppression) of a sign, symptom or condition of the disease) upon exposure to a microorganism (e.g., pathogen) capable of causing the disease. Immunity can be innate (e.g., non-adaptive (e.g., non-acquired) immune responses that exist in the absence of a previous exposure to an antigen) and/or acquired/adaptive (e.g., immune responses that are mediated by B and T cells following a previous exposure to antigen (e.g., that exhibit increased specificity and reactivity to the antigen)).

As used herein, the term “immunogen” refers to a molecule that stimulates a response from the adaptive immune system, which may include responses drawn from the group comprising an antibody response, a cytotoxic T cell response, a T helper response, and a T cell memory response. An immunogen may stimulate an upregulation of the immune response with a resultant inflammatory response, or may result in down regulation or immunosuppression. Typically, an immunogen is capable of initiating lymphocyte activation resulting in an antigen-specific immune response.

As use herein, the term “immunogenic composition” or “immunogenic formulation” refers to a preparation which, when administered to a vertebrate, especially an animal such as a mammal, will induce an immunogenic immune response, which may include responses that result in at least some level of immunity in the subject to which the immunogenic composition is administered.

The terms “linker” and “spacer” are used interchangeably herein to refer to a molecule or group of molecules (such as a monomer or polymer) that connects two molecules and often serves to place the two molecules in a desirable configuration. In some embodiments, the linker may be a covalent bond that connects two groups, or a chemical moiety having a chain of between 1 and 50 atoms in length, between 2 and 20 atoms in length, between 3 and 30 atoms in length, between 4 and 40 atoms in length, between 10 and 50 atoms in length, between 10 and 30 atoms in length, between 12 and 26 atoms in length, between 14 and 30 atoms in length, or any range therein in amounts of 1 atom in length. For example, a linker or a portion of a linker can have a length that is 1, 2, 3, 4, 5, 6, 8, 10, 12, 14, 16, 18, 20, 22, 24, 26, 28, 30, 32, 34, 36, 38, 40, 45, or 50 carbon atoms in length, where the linker may be linear, branched, cyclic, or a single atom. In some embodiments, the linker may be 10 angstroms, 11 angstroms, 12 angstroms, 13 angstroms, 14 angstroms, 15 angstroms, 16 angstroms, 17 angstroms, 18 angstroms, 19 angstroms, 20 angstroms, 22 angstroms, 23 angstroms, 24 angstroms, 25 angstroms, 26 angstroms, 27 angstroms, 28 angstroms, 29 angstroms, 30 angstroms, 32 angstroms, 34 angstroms, 36 angstroms, 38 angstroms, 40 angstroms, 45 angstroms, 50 angstroms, or any amount therein in increments of 1 angstrom, in length. In some embodiments, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, oligo(ethylene glycol); ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. In some embodiments, the linker may be at least one saccharide unit. A linker may be cleavable or non-cleavable.

As used herein, the term “moiety” refers to a portion of a molecule, which may be a functional group, a set of functional groups, and/or a specific group of atoms within a molecule, that is responsible for a characteristic chemical, biological, and/or medicinal property of the molecule.

The terms “monosaccharide unit” and “monosaccharide residue” are used interchangeably herein to refer to the most basic units of carbohydrates, and includes aldoses (e.g., D-glucose, D-galactose, D-mannose, D-ribose, D-arabinose, L-arabinose, D-xylose, etc.), ketoses (e.g., D-fructose, D-sorbose, D-tagatose, etc.), deoxysugars (e.g., L-rhamnose, L-fucose, etc.), deoxy-amino sugars (e.g., N-acetylglucosamine, N-acetylmannosamine, N-acetylgalactosamine, etc.), aldaric acids, uronic acids, ketoaldonic acids (e.g., sialic acid), ulsonic acids (e.g., KDO), or equivalents.

The term “monosaccharide derivative” or “monosaccharide unit derivative” is used herein to refer to a derivative of a monosaccharide in which a moiety of the monosaccharide is replaced with one or more substituents and/or functional groups, non-limiting examples of which include a deoxy sugar (alcoholic hydroxy group replaced by hydrogen), amino sugar (alcoholic hydroxy group replaced by amino group), a thio sugar (alcoholic hydroxy group replaced by thiol, or C═O replaced by C═S, or a ring oxygen of cyclic form replaced by sulfur), a seleno sugar, a telluro sugar, an aza sugar (ring carbon replaced by nitrogen), an amino sugar (ring oxygen replaced by nitrogen), a phosphano sugar (ring oxygen replaced with phosphorus), a phospha sugar (ring carbon replaced with phosphorus), a C-substituted monosaccharide (hydrogen at a non-terminal carbon atom replaced with carbon), an unsaturated monosaccharide, alditol (carbonyl group replaced with CHOH group), aldonic acid (aldehydic group replaced by carboxy group), a ketoaldonic acid, a uronic acid, an aldaric acid, and so forth. It is understood that a monosaccharide unit and the like can be further substituted.

The term “Neisseria” refers to a large genus of Gram negative bacteria that colonize the mucosal surfaces of many animals. Of the 11 species that colonize humans, only two are pathogens, N. meningitidis (one of the pathogens responsible for bacterial meningitis) and N. gonorrhoeae (the pathogen responsible for gonorrhea). In specific embodiments, infections that are targeted by the agents and compositions of the present disclosure include secondary infections which can arise from lack of treatment of a primary N. gonorrhoeae infection. Exemplary secondary infections include urethritis, dysuria, epididymitis, pelvic inflammatory disease, cervicitis and endometritis and also systemic gonococcal infections (e.g., those manifesting as arthritis, endocarditis or meningitis). The gonorrhea infection may be one caused by a strain of N. gonorrhoeae which is resistant to at least one known antibacterial drug, e.g. at least one β-lactam drug.

As used herein, the term “neutralize” and its grammatical equivalents refer to the ability of an antigen-binding molecule to inhibit activity of an antigen to which the antigen-binding molecule binds, including activity of an organism that expresses the antigen. The term “neutralizes” also includes within its scope causing a pathogenic organism that expresses the antigen to become non-pathogenic. For example, an organism may be neutralized through binding of an antigen-binding molecule that elicits an immune response (e.g., by phagocytic cells) against the organism. The organism may also be neutralized through binding of an antigen-binding molecule that disables the organism from reproducing, infecting a host, or producing a toxin.

The term “oligosaccharide” refers to a molecule comprising two or more, generally 2-10, typically 2-6, which are suitably glycosidically linked (e.g., by α- and/or β-glycosidic bonds), monosaccharide residues (also referred to herein as “monosaccharide units”). Oligosaccharides are considered to have a reducing end and a non-reducing end.

The term “oligosaccharide sequence” indicates that the monosaccharide residue/residues in the sequence are part of a larger glycoconjugate, which contains other monosaccharide residues in a chain, which may be branched, and/or may have substituted (e.g., natural substituted) modifications of oligosaccharide chains. The oligosaccharide chain is normally conjugated or otherwise linked to a lipid anchor or to a protein. In some embodiments, an oligosaccharide sequence is a non-reducing terminal oligosaccharide sequence, which means that the oligosaccharide sequence is not linked to another monosaccharide or oligosaccharide structure except optionally from the reducing end of the oligosaccharide sequence. The oligosaccharide sequence when present as a conjugate or chimeric molecule is suitably conjugated or otherwise linked from the reducing end of the oligosaccharide sequence, though other linkage positions which are tolerated by an antigen-binding molecule or other binding substance can also be used. It is to be understood that adjacent monosaccharide units defined in an oligosaccharide sequence of the present disclosure include within their scope alternative glycosidic linkages therebetween, and that specific monosaccharide units defined in a disclosed oligosaccharide sequence include within their scope derivatives of those monosaccharide units.

The term “operably linked” as used herein refers to a juxtaposition wherein the components so described are in a relationship permitting them to function in their intended manner. For example, “operably linking” a carrier to an oligosaccharide as described herein encompasses positioning and/or orientation of the carrier such that the oligosaccharide is presented to the immune system for the elicitation of an immune response to the oligosaccharide.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the disclosure include, but are not restricted to, any member of the subphylum Chordata including primates (e.g., humans, monkeys and apes, and includes species of monkeys such from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human, particularly a human in need of eliciting an immune response to a Neisseria organism. However, it will be understood that the aforementioned terms do not imply that symptoms are present.

By “pharmaceutically acceptable vehicle” is meant a solid or liquid filler, diluent or encapsulating substance that can be safely used in topical or systemic administration to an animal, preferably a mammal, including humans. Representative pharmaceutically acceptable vehicles include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated. The term “vehicle” typically denotes an organic or inorganic ingredient, natural or synthetic, with which the active agent combined to facilitate the application.

The term “pharmaceutically acceptable salt” as used herein is a broad term, and is to be given its ordinary and customary meaning to a person of ordinary skill in the art (and is not to be limited to a special or customized meaning), and refers without limitation to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17.sup.th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

“Polypeptide”, “peptide”, “protein” and “proteinaceous molecule” are used interchangeably herein to refer to molecules comprising or consisting of a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

The term “resistant” is intended to refer to strains of bacteria that have shown non-susceptibility to one or more known antibacterial drugs. A non-susceptible strain is one in which the MIC of a given compound or class of compounds for that strain has shifted to a higher number than for corresponding susceptible strains. For example, it may refer to strains that are non-susceptible to β-lactam antibiotics, strains that are non-susceptible to one or more fluoroquinolones and/or strains that are non-susceptible to one or more other antibiotics (i.e., antibiotics other than β-lactams and fluoroquinolones). In certain embodiments, the term “resistant” may refer to one in which the MIC of a given compound or class of compounds for that strain has shifted to a significantly higher number than for corresponding susceptible strains. A bacterial strain might be said to be resistant to a given antibiotic when it is inhibited in vitro by a concentration of this drug that is associated with a high likelihood of therapeutic failure. The bacterial strain may be resistant to one or more fluoroquinolone antibiotics, e.g., one or more antibiotics selected from levofloxacin, enoxacin, fleroxacin, lomefloxacin, nadifloxacin, norfloxacin, rufloxacin, balofloxacin, grepafloxacin, pazufloxacin, sparfloxacin, temafloxacin, tosufloxacin, besifloxacin, clinafloxacin, garenoxacin, gemifloxacin, gatifloxacin, sitafloxacin, trovafloxacin, prulifloxacin, ciprofloxacin, pefloxacin, moxifloxacin, ofloxacin, delafloxacin, zabofloxacin, avarofloxacin, finafloxacin.

As used herein, the term “saccharide unit” refers to a single monosaccharide residue/moiety.

As used herein, the term “terminal saccharide unit” refers to the saccharide unit that is linked to either none (in case of a monosaccharide) or only one neighboring saccharide unit (in case of an oligosaccharide).

By the terms “treat”, “treating” or “treatment of” (or grammatically equivalent terms) it is meant that the severity of a subject's condition is reduced or at least partially improved or ameliorated and/or that some alleviation, mitigation or decrease in at least one clinical symptom is achieved and/or there is a delay in the progression of the condition.

Each embodiment described herein is to be applied mutatis mutandis to each and every embodiment unless specifically stated otherwise.

### 2. Abbreviations

The following abbreviations are used throughout the application:
CFU=colony forming units
CMP=cytidine monophosphate
CRM197=non-toxic mutant of diphtheria toxin
EDC=1-ethyl-3-(3-dimethylaminopropyl)carbodiimide
Gal=galactose
Glc=glucose
GlcNAc=N-acetylglucosamine
Hep=heptose
hr.=hour
HRP=horseradish peroxidase
Ig=immunoglobulin
IgG=immunoglobulin G
IgG1=immunoglobulin G1
IgG2a=immunoglobulin G2a
IgG2b=immunoglobulin G2b
IgG3=immunoglobulin G3
IgG4=immunoglobulin G4
IgM=immunoglobulin M
kDa=kilodaltons(s)
KDO=3-deoxy-D-manno-oct-2-ulosonic acid, keto-deoxyoctonic acid, or keto-deoxyoctulosonate
LNnT=lacto-N-neotetraose
LOS=lipooligosaccharide
MAb=monoclonal antibody
MDR=multidrug resistant
MIC=minimum inhibitory concentration
Neu5Ac=N-acetylneuraminic acid
NCS=N-chlorosuccinimide
NHS=N-hydroxysuccinimide
NTHi=non-typeable Haemophilus influenzae
OMV=outer membrane vesicle
PMN=polymorphonuclear leukocyte
TMB=3,3′, 5,5 tetramethylbenzidine

### 3. Chimeric Molecules Comprising Neisseria Oligosaccharides

The present disclosure is based in part on the determination that Neisseria LOS alpha-2,3-sialyltransferase (Lst) is present in the cytoplasm of the bacterium and that in addition to transferring Neu5Ac from CMP-Neu5Ac to the terminus of LOS, Lst is able to transfer KDO to LOS in place of Neu5Ac in both N. gonorrhoeae and N. meningitidis. Notably, the present inventors have found that KDO is expressed as a terminal LOS structure in in vivo samples from infected women and that the anti-KDO monoclonal antibody 6E4 can mediate opsonophagocytic killing of N. gonorrhoeae. Accordingly, it is proposed that KDO expressed on gonococcal LOS represents a new oligosaccharide antigen for the development of immunogenic compositions against Neisseria organisms.

Accordingly, the present disclosure relates to chimeric molecules for use in eliciting an immune response to Neisseria organisms such as N. gonorrhoeae or N. meningitidis and methods of using such chimeric molecules for treating and/or inhibiting Neisseria infection, for example by eliciting an immune response targeting Neisseria oligosaccharide antigens.

The chimeric molecules comprise a carrier and an oligosaccharide that comprises an oligosaccharide sequence corresponding to a Neisseria LOS, wherein the oligosaccharide sequence comprises KDO as a terminal saccharide unit. In specific embodiments, the chimeric molecule is represented by formula (I):

{[KDO-OS]ₙ-L}ₘ-Cr (I)
wherein:
KDO-OS represents an oligosaccharide sequence comprising 2-10 (e.g., 2, 3, 4, 5, 6, 7, 8, 9 or 10), typically 2-6, monosaccharide units and KDO as a terminal saccharide unit;
L is an optional linker;
Cr is a carrier;
n is 1 to 4; and
m is at least 1,
or a pharmaceutically acceptable salt thereof.

The oligosaccharide sequence preferably comprises KDO attached to the Gal, suitably wherein the Gal is further attached to GlcNAc. In representative embodiments, the KDO is attached to Gal via an α(2—>3) linkage and/or the Gal is attached to GlcNAc via a β(1→4) linkage. In non-limiting examples of this type, the oligosaccharide sequence comprises KDOα(2→3)Gal, or KDOα(2→3)Galβ(1→4)GlcNAc.

Suitable carriers are known in the art (See e.g., Remington's Pharmaceutical Sciences (18th ed., Mack Easton, Pa. (1990)) and may include, for example, proteins, peptides, lipids, polymers, dendrimers, virosomes, virus-like particles (VLPs), outer membrane proteins (OMPs) or combinations thereof, which by themselves may not display particular antigenic properties, but can support immunogenic reaction of a host to the oligosaccharides of the present disclosure (antigens) displayed on the surface of the carrier(s).

In specific embodiments, the carrier is a protein carrier, including but not limited to, bacterial toxoids, toxins, exotoxins, and nontoxic derivatives thereof, such as tetanus toxoid, tetanus toxin Fragment C, diphtheria toxoid, CRM (a nontoxic diphtheria toxin mutant) such as CRM 197, cholera toxoid, S. aureus exotoxins or toxoids, E coli heat labile enterotoxin, P. aeruginosa exotoxin A, including recombinantly produced, genetically detoxified variants thereof; bacterial outer membrane proteins, such as N. meningitidis serotype B outer membrane protein complex (OMPC), outer membrane class 3 porin (rPorB) and other porins; keyhole limpet hemocyanine (KLH), hepatitis B virus core protein, thyroglobulin, albumins, such as bovine serum albumin (BSA), human serum albumin (HSA), and ovalbumin; pneumococcal surface protein A (PspA), pneumococcal adhesin protein (PsaA); purified protein derivative of tuberculin (PPD); transferrin binding proteins, polyamino acids, such as poly(lysine:giutamic acid); peptidylagonists of TLR-5 (e.g., flagellin of motile bacteria like Listeria); and derivatives and/or combinations of the above carriers. Preferred carriers for use in humans include tetanus toxoid, CRM 197, and OMPC.

Depending on the type of bonding between the linker and the carrier, and the structural nature of the carrier and oligosaccharide, a carrier may display on average, for example, 1 to 500, 1 to 100, 1 to 20, or 3 to 9 oligosaccharide units on its surface.

Methods for attaching an oligosaccharide to a carrier, such as a carrier protein are conventional, and a skilled practitioner can create conjugates in accordance with the present disclosure using conventional methods. Guidance is also available in various disclosures, including, for example, U.S. Pat. Nos. 4,356,170; 4,619,828; 5,153,312; 5,422,427; and 5,445,817; and in various print and online Pierce protein cross-linking guides and catalogs (Thermo Fisher, Rockford, Ill.)

Following conjugation of the oligosaccharide to the carrier, the chimeric molecule can be purified by a variety of techniques well known to one of skill in the art. One goal of the purification step is to remove the unbound oligosaccharide from the conjugation reaction product composition. An illustrative method for purification involves ultrafiltration in the presence of ammonium sulfate, which is described in U.S. Pat. No. 6,146,902. Alternatively, the chimeric molecules can be purified away from unreacted oligosaccharide and carrier by any number of standard techniques including, for example, size exclusion chromatography, density gradient centrifugation, hydrophobic interaction chromatography, or ammonium sulfate fractionation (see, for example, Anderson et al., 1986. J. Immunol. 137:1181-1186; and Jennings & Lugowski, 1981. J. Immunol. 127:1011-1018). The compositions and purity of the chimeric molecules can be determined by GLC-MS and MALDI-TOF spectrometry.

In certain embodiments, the oligosaccharide antigens of the present disclosure are conjugated to CRM197, a commercially available protein carrier used in a number of FDA approved vaccines. CRM-conjugates have the advantage of being easier to synthesize, purify and characterize than other FDA approved carriers such as OMPC. Oligosaccharide antigens may be conjugated to CRM via thiol-bromoacetyl conjugation chemistry. CRM activation may be achieved by reacting the lysine side chains with the NHS ester of bromoacetic acid using standard conditions as previously described in U.S. Pat. Appl. Publ. 2007-0134762. CRM may be functionalized with 10-20 bromoacetyl groups per protein (n=10-20) prior to conjugation. Conjugation may be performed at pH=9 to avoid aggregation of CRM. Careful monitoring of pH must be employed to ensure complete CRM reaction with NHS-bromoacetate while minimizing background hydrolysis of CRM. Activated CRM may be purified by size exclusion chromatography prior to conjugation. Antigen-CRM conjugates may be synthesized by reacting thiol-terminated carbohydrate antigens with bromoacetamide-activated CRM.

CRM conjugates may be purified via size exclusion chromatography to remove and recover any unreacted carbohydrate. MBTH (specific for GlcNAc residues) and Bradford assays may be used to determine carbohydrate:protein ratio and protein content, respectively, as previously described (see, for example, Manzi et al., 1995. Curr. Prot. Mol. Biol., section 17.9.1 (Suppl. 32)). In specific embodiments, a minimum oligosaccharide content of about 10% by weight for each conjugate may be generated. Typically, a conjugate may include about 3-20 antigens per protein carrier.

In other embodiments, oligosaccharide antigens may be conjugated to one or more carriers suitable for development of diagnostic assays, including ELISAs and microarrays. Exemplary carriers for use in such assays include bovine serum albumin (BSA), keyhole limpet hemocyanine (KLH), biotin, a label, a glass slide or a gold surface. By way of example, synthetic carbohydrate antigens may be conjugated to BSA by a thiol-maleimide coupling procedure (FIG. 5B). Maleimide-BSA contains 15-20 maleimide groups per protein (n=15-20). Accordingly, oligosaccharide antigens may be conjugated to maleimide functionalized BSA, whereby a 20-fold molar excess of the antigen is reacted with commercially available Imject™ maleimide BSA (Pierce) in maleimide conjugation buffer (Pierce). Conjugation may be performed at pH=7.2 to avoid hydrolysis of the maleimide group during conjugation.

BSA conjugates may be purified via size exclusion chromatography to remove and recover any unreacted carbohydrate. Characterization via the phenol-sulfuric acid and Bradford assays may be performed along with MALDI-MS to provide information on the carbohydrate content and valency of the conjugates. In preferred embodiments, conjugates will contain a minimum oligosaccharide content of about 10% by weight per BSA conjugate and >8 antigen copies per conjugate.

In a related aspect, the disclosure relates to an oligosaccharide that comprises an oligosaccharide sequence corresponding to a Neisseria LOS, wherein the oligosaccharide sequence comprises KDO as a terminal saccharide unit, as broadly described above and elsewhere herein.

### 4. Antigen-Binding Molecules

The oligosaccharides disclosed herein are structural mimetics of Neisseria LOS and in accordance with the present disclosure are useful for producing antigen-binding molecules that bind specifically with Neisseria organisms, including pathogenic Neisseria organisms such as gonorrhoeae and N. meningitidis.

Those of ordinary skill in the art will appreciate the well-developed knowledge base on antigen-binding proteins such as set forth, for example, in Abbas et al., Cellular and Molecular Immunology, 6th ed., W.B. Saunders Company (2010) or Murphey et al., Janeway's Immunobiology, 8th ed., Garland Science (2011). In some embodiments, antigen binding proteins that bind specifically with a Neisseria LOS are antibodies. Antibodies include intact antibodies and antigen binding fragments thereof, as described in the definition section. An antibody may comprise a complete antibody molecule (including polyclonal, monoclonal, chimeric, humanized, or human versions having full length heavy and/or light chains), or comprise an antigen binding fragment thereof. Antibody fragments include F(ab′)2, Fab, Fab′, Fv, Fc, and Fd fragments, and can be incorporated into single domain antibodies, single-chain antibodies, maxibodies, minibodies, intrabodies, diabodies, triabodies, tetrabodies, v-NAR and bis-scFv (see e.g., Hollinger and Hudson, 2005, Nature Biotechnology, 23, 9, 1126-1136). Also included are antibody polypeptides such as those disclosed in U.S. Pat. No. 6,703,199, including fibronectin polypeptide monobodies. Other antibody polypeptides are disclosed in U.S. Patent Publication 2005/0238646, which are single-chain polypeptides.

Numerous methods of preparing antibodies to antigens of interest are known in the art. For example, monoclonal antibodies that bind specifically with a Neisseria LOS can be made using conventional hybridoma methods that are often based on the seminal method of Kohler, G. et al. (1975, “Continuous Cultures Of Fused Cells Secreting Antibody Of Predefined Specificity,” Nature 256:495-497) or a modification thereof. Typically, monoclonal antibodies are developed in non-human species, such as mice. In general, a mouse or rat is used for immunization but other animals may also be used. The antibodies may be produced by immunizing mice with an immunogenic amount of an immunogen, in this case a chimeric molecule of the present disclosure. The immunogen may be administered multiple times at periodic intervals such as, bi weekly, or weekly, or may be administered in such a way as to maintain viability in the animal.

To monitor the antibody response, a small biological sample (e.g., blood) may be obtained from the animal and tested for antibody titer against the immunogen. The spleen and/or several large lymph nodes can be removed and dissociated into single cells. If desired, the spleen cells may be screened (after removal of non-specifically adherent cells) by applying a cell suspension to a plate or to a well coated with the antigen. B-cells, expressing membrane-bound immunoglobulin specific for the antigen, will bind to the plate, and are not rinsed away with the rest of the suspension. Resulting B-cells, or all dissociated spleen cells, can then be fused with myeloma cells (e.g., X63-Ag8.653 and those from the Salk Institute, Cell Distribution Center, San Diego, Calif.). Polyethylene glycol (PEG) may be used to fuse spleen or lymphocytes with myeloma cells to form a hybridoma. The hybridoma is then cultured in a selective medium (e.g., hypoxanthine, aminopterin, thymidine medium, otherwise known as “HAT medium”). The resulting hybridomas are then plated by limiting dilution, and are assayed for the production of antibodies that bind specifically to the immunogen, using, for example, FACS (fluorescence activated cell sorting) or immunohistochemistry (IHC) screening. The selected monoclonal antibody-secreting hybridomas are then cultured either in vitro (e.g., in tissue culture bottles or hollow fiber reactors), or in vivo (e.g., as ascites in mice).

As another alternative to the cell fusion technique, Epstein-Barr Virus (EBV)-immortalized B cells may be used to produce monoclonal antibodies that bind specifically with a Neisseria LOS. The hybridomas are expanded and subcloned, if desired, and supernatants are assayed for anti-immunogen activity by conventional assay procedures (e.g., FACS, IHC, radioimmunoassay, enzyme immunoassay, fluorescence immunoassay, etc.).

Thus, the present disclosure further contemplates methods of producing an antigen-binding molecule that binds specifically with a Neisseria organism, wherein the method comprises: (1) immunizing an animal with a chimeric molecule as disclosed herein; (2) detecting a B cell from the animal, which binds specifically with the chimeric molecule, or Neisseria organism or LOS thereof; and (3) isolating the antigen-binding molecule expressed by that B cell.

The present disclosure also encompasses antigen-binding molecule that are produced by such methods as well as derivatives thereof. Also encompassed are cells including hybridomas that are capable of producing the antigen-binding molecules of the disclosure, and methods of producing antigen-binding molecules from those cells. In specific embodiments, the antigen-binding molecules produced by the methods and cells of the disclosure are preferably neutralizing antigen-binding molecules.

Also contemplated are chimeric antibodies and humanized antibodies. In some embodiments, a humanized monoclonal antibody comprises the variable domain of a murine antibody (or all or part of the antigen binding site thereof) and a constant domain derived from a human antibody. Alternatively, a humanized antibody fragment may comprise the antigen binding site of a murine monoclonal antibody and a variable domain fragment (lacking the antigen-binding site) derived from a human antibody. Procedures for the production of engineered monoclonal antibodies include those described in Riechmann et al., 1988, Nature 332:323, Liu et al., 1987, Proc. Nat. Acad. Sci. USA 84:3439, Larrick et al., 1989, Bio/Technology 7:934, and Winter et al., 1993, TIPS 14:139. In one embodiment, the chimeric antibody is a CDR grafted antibody. Techniques for humanizing antibodies are discussed in, e.g., U.S. Pat. Nos. 5,869,619; 5,225,539; 5,821,337; 5,859,205; 6,881,557, Padlan et al., 1995, FASEB J. 9:133-39, Tamura et al., 2000, J. Immunol. 164:1432-41, Zhang, W., et al., Molecular Immunology 42(12):1445-1451, 2005; Hwang W. et al., Methods 36(1):35-42, 2005; Dall'Acqua W F, et al., Methods 36(1):43-60, 2005; and Clark, M., Immunology Today 21(8):397-402, 2000.

An antibody of the present disclosure may also be a fully human monoclonal antibody. Fully human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein.

Procedures have been developed for generating human monoclonal antibodies in non-human animals. For example, mice in which one or more endogenous immunoglobulin genes have been inactivated by various means have been prepared. Human immunoglobulin genes have been introduced into the mice to replace the inactivated mouse genes. In this technique, elements of the human heavy and light chain locus are introduced into strains of mice derived from embryonic stem cell lines that contain targeted disruptions of the endogenous heavy chain and light chain loci (see also Bruggemann et al., 1997. Curr. Opin. Biotechnol. 8:455-58). For example, human immunoglobulin transgenes may be mini-gene constructs, or transloci on yeast artificial chromosomes, which undergo B-cell-specific DNA rearrangement and hypermutation in the mouse lymphoid tissue.

Antibodies produced in the animal incorporate human immunoglobulin polypeptide chains encoded by the human genetic material introduced into the animal. In one embodiment, a non-human animal, such as a transgenic mouse, is immunized with a subject chimeric polypeptide or complex immunogen.

Examples of techniques for production and use of transgenic animals for the production of human or partially human antibodies are described in U.S. Pat. Nos. 5,814,318, 5,569,825, and 5,545,806, Davis et al., Production of human antibodies from transgenic mice in Lo, ed. Antibody Engineering: Methods and Protocols, Humana Press, NJ: 191-200 (2003), Kellermann et al., Curr Opin Biotechnol. 2002, 13:593-97, Russel et al., Infect Immun. 2000, 68:1820-26, Gallo et al., Eur J. Immun. 2000, 30:534-40, Davis et al., Cancer Metastasis Rev. 1999, 18:421-25, Green, J Immunol Methods 1999, 231:11-23, Jakobovits, Advanced Drug Delivery Reviews 1998, 31:33-42, Green et al., J Exp Med. 1998, 188:483-95, Jakobovits A, Exp. Opin. Invest. Drugs 1998, 7:607-14, Tsuda et al., Genomics 1997, 42:413-21, Mendez et al., Nat. Genet. 1997, 15:146-56, Jakobovits, Curr Biol. 1994, 4:761-63, Arbones et al., Immunity 1994, 1:247-60, Green et al., Nat. Genet. 1994, 7:13-21, Jakobovits et al., Nature 1993, 362:255-58, Jakobovits et al., Proc Natl Acad Sci USA 1993, 90:2551-55. Chen, J., M. et al. Int. Immunol. 1993, 5: 647-656, Choi et al., Nature Genetics 1993, 4: 117-23, Fishwild et al., Nature Biotech. 1996, 14: 845-51, Harding et al., 1995, Annals of the New York Academy of Sciences, Lonberg et al., 1994, Nature 368: 856-59, Lonberg, 1994, Transgenic Approaches to Human Monoclonal Antibodies in Handbook of Experimental Pharmacology 113: 49-101, Lonberg et al., Int. Rev. Immunol. 1995, 13: 65-93, Neuberger, Nature Biotech. 1996, 14: 826, Taylor et al., Nucleic Acids Research 1992, 20: 6287-95, Taylor et al., Int. Immunol. 1994, 6: 579-91, Tomizuka et al., Nature Genetics 1997, 16: 133-43, Tomizuka et al., Proc Natl Acad Sci USA 2000, 97: 722-27, Tuaillon et al., Proc Natl Acad Sci USA 1993, 90: 3720-24, and Tuaillon et al., J. Immunol. 1994, 152: 2912-20; Lonberg et al., Nature 1994, 368:856; Taylor et al., Int. Immunol. 1994, 6:579; U.S. Pat. No. 5,877,397; Bruggemann et al., Curr. Opin. Biotechnol. 1997 8:455-58; Jakobovits et al., Ann. N.Y. Acad. Sci. 1995. 764:525-35. In addition, protocols involving the XenoMouse®. (Abgenix, now Amgen, Inc.) are described, for example in U.S. 05/0118643 and WO 05/694879, WO 98/24838, WO 00/76310, and U.S. Pat. No. 7,064,244.

Alternatively, the chimeric molecules disclosed herein may be used to screen for antigen-binding molecules from antigen-binding molecule libraries. For example, a chimeric molecule of the present disclosure may be immobilized to a solid support (e.g., a silica gel, a resin, a derivatized plastic film, a glass bead, cotton, a plastic bead, a polystyrene bead, an alumina gel, or a polysaccharide, a magnetic bead), and screened for binding to antigen-binding molecules. As an alternative, the antigen-binding molecules may be immobilized to a solid support and screened for binding to the chimeric molecule. Any screening assay, such as a panning assay, ELISA, surface plasmon resonance, or other antigen-binding molecule screening assay known in the art may be used to screen for antigen-binding molecules that bind to a chimeric molecule disclosed herein, particularly its oligosaccharide component. The antigen-binding molecule library screened may be a commercially available library, an in vitro generated library, or a library obtained by identifying and cloning or isolating antibodies from an individual infected with a Neisseria organism. In particular embodiments, the antigen-binding molecule library is generated from a subject that has been infected with a Neisseria organism. Antigen-binding molecule libraries may be generated in accordance with methods known in the art. In a particular embodiment, the library is generated by cloning the antibodies and using them in phage display libraries or a phagemid display library.

The present disclosure further encompasses fragments of an anti-Neisseria LOS antibody. Such fragments can consist entirely of antibody-derived sequences or can comprise additional sequences. Examples of antigen-binding fragments include Fab, F(ab′)2, single chain antibodies, diabodies, triabodies, tetrabodies, and domain antibodies. Other examples are provided in Lunde et al., Biochem. Soc. Trans. 2002, 30:500-06.

Single chain antibodies may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (VL and VH). The resulting polypeptides can fold back on themselves to form antigen-binding monomers, or they can form multimers (e.g., dimers, trimers, or tetramers), depending on the length of a flexible linker between the two variable domains (Kortt et al., Prot. Eng. 1997, 10:423; Kortt et al., Biomol. Eng. 2001, 18:95-108). By combining different VL and VH-comprising polypeptides, one can form multimeric scFvs that bind to different epitopes (Kriangkum et al., Biomol. Eng. 2001, 18:31-40). Techniques developed for the production of single chain antibodies include those described in U.S. Pat. No. 4,946,778; Bird, Science 1988, 242:423; Huston et al., Proc. Natl. Acad. Sci. USA 1988, 85:5879; Ward et al., Nature 1989, 334:544, de Graaf et al., Methods Mol. Biol. 2002, 178:379-87.

Antigen binding fragments derived from an antibody can also be obtained, for example, by proteolytic hydrolysis of the antibody, for example, pepsin or papain digestion of whole antibodies according to conventional methods. By way of example, antibody fragments can be produced by enzymatic cleavage of antibodies with pepsin to provide a 5S fragment termed F(ab′)2. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using papain produces two monovalent Fab fragments and an Fc fragment directly. These methods are described, for example, by Goldenberg, U.S. Pat. No. 4,331,647, Nisonoff et al., Arch. Biochem. Biophys. 1960, 89:230; Porter, Biochem. J. 1959, 73:119; Edelman et al., in Methods in Enzymology 1:422 (Academic Press 1967); and by Andrews, S. M. and Titus, J. A. in Current Protocols in Immunology (Coligan J. E., et al., eds), John Wiley & Sons, New York (2003), pages 2.8.1-2.8.10 and 2.10A.1-2.10A.5. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the antigen that is recognized by the intact antibody.

Another form of an antibody fragment is a peptide comprising one or more complementarity determining regions (CDRs) of an antibody. CDRs can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology 2:106, 1991; Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)). The antibody fragment further may comprise at least one variable region domain of an antibody described herein. Thus, for example, the V region domain may be monomeric and be a VL and VH domain, which is capable of independently binding a subject ectodomain polypeptide or complex with an affinity at least equal to 10⁻⁷ M or less.

The variable region domain may be any naturally occurring variable domain or an engineered version thereof. By engineered version is meant a variable region domain that has been created using recombinant DNA engineering techniques. Such engineered versions include those created, for example, from a specific antibody variable region by insertions, deletions, or changes in or to the amino acid sequences of the specific antibody. Particular examples include engineered variable region domains containing at least one CDR and optionally one or more framework amino acids from a first antibody and the remainder of the variable region domain from a second antibody.

The variable region domain may be covalently attached at a C-terminal amino acid to at least one other antibody domain or a fragment thereof. Thus, for example, a VH domain that is present in the variable region domain may be linked to an immunoglobulin CH1 domain, or a fragment thereof. Similarly a VL domain may be linked to a CK domain or a fragment thereof. In this way, for example, the antibody may be a Fab fragment wherein the antigen binding domain contains associated VH and VL domains covalently linked at their C-termini to a CH1 and CK domain, respectively. The CH1 domain may be extended with further amino acids, for example to provide a hinge region or a portion of a hinge region domain as found in a Fab′ fragment, or to provide further domains, such as antibody CH2 and CH3 domains.

Antigen-binding molecules identified in the methods described herein may be tested for neutralizing activity and lack of autoreactivity using biological assays known in the art or described herein. In some embodiments, an antigen-binding molecule elicited or identified using a chimeric molecule disclosed herein, neutralizes a Neisseria organism.

In some embodiments, antigen-binding molecules elicited or identified using a chimeric molecule disclosed herein may be used to monitor the efficacy of a therapy and/or disease progression.

Antigen-binding molecules elicited or identified using a chimeric molecule may be used in diagnostic immunoassays to detect the presence of a Neisseria organism in biological samples, passive immunotherapy, and generation of antiidiotypic antigen-binding molecules. In addition, the ability of the antigen-binding molecules to neutralize a Neisseria organism and the specificity of the antigen-binding molecules for the Neisseria LOS may be tested prior to using the antigen-binding molecules in passive immunotherapy.

Specific binding of an antigen-binding molecule to chimeric molecule disclosed herein, particularly its oligosaccharide component, and cross-reactivity with other antigens can be assessed by any method known in the art. Immunoassays which can be used to analyze specific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York).

In some embodiments, the antigen-binding molecules disclosed herein are used in immunodetection methods for binding, purifying, removing, quantifying and otherwise generally detecting Neisseria organisms such as N. gonorrhoeae or N. meningitidis. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The immunodetection methods also include methods for detecting and quantifying the amount of Neisseria organism or related components (e.g., LOS thereof) in a sample and the detection and quantification of any immune complexes formed during the binding process. In non-limiting examples, a sample suspected of containing a Neisseria organism is obtained from a patient, and the sample is contacted with an antigen-binding molecule that binds specifically to the oligosaccharide component of a chimeric molecule disclosed herein, followed by detecting and quantifying the amount of immune complexes formed under the specific conditions. In terms of antigen detection, the biological sample analyzed may be any sample that is suspected of containing a Neisseria organism, such as a tissue section or specimen, a homogenized tissue extract, a biological fluid, including a biological fluid and/or tissue associated with infection by a Neisseria organism.

Contacting the chosen biological sample with the antigen-binding molecule under suitable conditions and for a period of time sufficient to allow the formation of immune complexes (primary immune complexes) is generally a matter of simply adding the antigen-binding molecule to the sample and incubating the mixture for a period of time long enough for the antigen-binding molecule to form immune complexes with, i.e., to bind a Neisseria organism or related components (e.g., LOS thereof) present in the sample. After this time, the sample-antigen-binding molecule composition, such as a tissue section, ELISA plate, dot blot or Western blot, will generally be washed to remove any non-specifically bound antigen-binding molecule species, allowing only those antigen-binding molecule specifically bound within the primary immune complexes to be detected.

### 5. Compositions

The chimeric molecules and antigen-binding molecules disclosed herein (“active agents”) may be included in pharmaceutical compositions (including therapeutic and prophylactic formulations), typically combined together with one or more pharmaceutically acceptable vehicles and, optionally, other therapeutic ingredients (e.g., antimicrobials, immunogens, etc.).

Pharmaceutically-acceptable vehicles may be employed to enhance the delivery and/or control the duration of action of the active agent. Control release preparations may be achieved through the use of polymers to complex or absorb the active agent, and/or adjuvants. Controlled delivery may be effected by selecting appropriate macromolecules (for example polyesters, polyamino acids, polyvinyl, pyrrolidone, ethylenevinylacetate, methylcellulose, carboxymethylcellulose, or protamine sulfate) and the concentration of macromolecules as well as the method of incorporation in order to control release. Another possible method to control the duration of action by controlled release preparations is to incorporate an active agent disclosed herein into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, interfacial polymerization, for example, hydroxymethylcellulose or gelatin-microcapsules and poly(methylmethacylate)-microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences, supra.

Adjuvants can be included in the compositions. Exemplary adjuvants and/or adjuvant combinations may be selected from mineral salts, including aluminum salts, such as aluminum phosphate and aluminum hydroxide (alum) (e.g., Alhydrogel™, Superfos, Denmark) and calcium phosphate; RIBI, which contains three components extracted from bacteria, monophosphoryl lipid A, trehalosedimycolate, and cell wall skeleton (MPL+TDM+CWS) in a 2% squalene/Tween 80 emulsion, whereby any of the 3 components MPL, TDM or CWS may also be used alone or combined 2 by 2; toll-like receptor (TLR) agonists, including, for example, agonists of TLR-1 (e.g. tri-acyl lipopeptides); agonists of TLR-2 [e.g. peptidoglycan of gram-positive bacteria like streptococci and staphylococci; lipoteichoic acid]; agonists of TLR-3 (e.g. double-stranded RNA and their analogs such as poly 1:C); agonists of TLR-4 (e.g. lipopolysaccharide (endotoxin) of gram-negative bacteria like Salmonella and E. coli); agonists of TLR-5 (e.g. flagellin of motile bacteria like Listeria); agonists of TLR-6 (e.g. with TLR-2 peptidoglycan and certain lipids (diacyllipopeptides)); agonists of TLR-7 (e.g. single-stranded RNA (ssRNA) genomes of such viruses as influenza, measles, and mumps; and small synthetic guanosine-base antiviral molecules like loxoribine and ssRNA and their analogs); agonists of TLR-8 (e.g. binds ssRNA); agonists of TLR-9 (e.g. unmethylatedCpG of the DNA of the pathogen and their analogs; agonists of TLR-10 (function not defined) and TLR-11—(e.g. binds proteins expressed by several infectious protozoans (Apicomplexa), specific toll-like receptor agonists include monophosphoryl lipid A (MPL®), 3 De-O-acylatedmonophosphoryl lipid A (3 D-MPL), OM-174 (E. coli lipid A derivative); OM triacyl lipid A derivative, and other MPL- or lipid A-based formulations and combinations thereof, including MPL®-SE, RC-529 (Dynavax Technologies), AS01 (liposomes+MPL+QS21), AS02 (oil-in-water PL+QS-21), and AS04 (Alum+MPL)(GlaxoSmith Kline, Pa.), CpG-oligodeoxynucleotides (ODNs) containing immunostimulatory CpG motifs, double-stranded RNA, polyinosinic:polycytidylic acid (poly I:C), and other oligonucleotides or polynucleotides optionally encapsulated in liposomes; oil-in-water emulsions, including AS03 (GlaxoSmith Kline, Pa.), MF-59 (microfluidized detergent stabilized squalene oil-in-water emulsion; Novartis), and Montanide ISA-51 VG (stabilized water-in-oil emulsion) and Montanide ISA-720 (stabilized water/squalene; Seppic Pharmaceuticals, Fairfield, N.J.); cholera toxin B subunit; saponins, such as Quil A or QS21, an HPLC purified non-toxic fraction derived from the bark of QuillajaSaponaria Molina (STIMULON™ (Antigenics, Inc., Lexington, Mass.) and saponin-based adjuvants, including immunostimulating complexes (ISCOMs; structured complex of saponins and lipids) and other ISCOM-based adjuvants, such as ISCOMATRIX™ and AbISCO®-100 and -300 series adjuvants (Isconova AB, Uppsala, Sweden); QS21 and 3 D-MPL together with an oil in water emulsion as disclosed in U.S. Pat. Appl. No. 2006/0073171; stearyl tyrosine (ST) and amide analogs thereof; virus-like particles (VLPs) and reconstituted influenza virosomes (IRIVs); complete Freund's adjuvant (CFA); incomplete Freund's adjuvant (IFA); E. coli heat-labile enterotoxin (LT); immune-adjuvants, including cytokines, such as IL-2, IL-12, GM-CSF, Flt3, accessory molecules, such as 67.1, and mast cell (MC) activators, such as mast cell activator compound 48/80 (C48/80); water-insoluble inorganic salts; liposomes, including those made from DNPC/Chol and DC Chol; micelles; squalene; squalane; muramyl dipeptides, such as N-acetyl-muramyl-L-threonyl-D-isoglutamine (thr-MDP) as found in U.S. Pat. No. 4,606,918, N-acetyl-normuramyl-L-alanyl-D-isoglutamine (nor-MDP), and N-acetylmuramyl-L-alanyl-D-isoglutaminyl-L-alanine-2-(1′2′-dipalmitoyl-n-glycero-3-hydroxyphosphoryl; SAF-1 (Syntex); AS05 (GlaxoSmith Kline, Pa.); and combinations thereof.

In some embodiments, adjuvant potency may be enhanced by combining multiple adjuvants as described above, including combining various delivery systems with immunopotentiating substances to form multi-component adjuvants with the potential to act synergistically to enhance antigen-specific immune responses in vivo. Exemplary immunopotentiating substances include the above-described adjuvants, including, for example, MPL and synthetic derivatives, MDP and derivatives, oligonucleotides (CpGetc), ds RNAs, alternative pathogen-associated molecular patterns (PAMPs) (E. coli heat labile enterotoxin; flagellin, saponins (QS-21 etc), small molecule immune potentiators (SMIPs, e.g., resiquimod [R848]), cytokines, and chemokines.

The compositions of the present disclosure can be formulated according to known methods to prepare pharmaceutically useful compositions, whereby these materials, or their functional derivatives, are combined in admixture with a pharmaceutically acceptable vehicle (or diluents). Suitable vehicles and their formulation, inclusive of other human proteins, e.g., human serum albumin, are described, for example, in Remington's Pharmaceutical Sciences, supra. In order to form a pharmaceutically acceptable composition suitable for effective administration, such compositions will contain an effective amount of the above-described compounds together with a suitable amount of protein carrier and/or vehicle.

Typically, the compositions may be prepared as injectables, either as liquid solutions or suspensions; solid forms suitable for solution in, or suspension in, liquid vehicles prior to injection. An aqueous composition for parenteral administration, for example, may include a solution of the active agent(s) dissolved or suspended in a pharmaceutically acceptable vehicle or diluent, preferably a primarily aqueous vehicle. Pharmaceutically acceptable vehicles or diluents may include water, saline, including neutral saline solutions buffered with phosphate, Tris, glycerol, ethanol, and the like. An aqueous composition may be formulated as a sterile, pyrogen-free buffered saline or phosphate-containing solution, which may include a preservative or may be preservative free. Suitable preservatives include benzyl alcohol, parabens, thimerosal, chlorobutanol, and benzalkonium chloride, for example. Aqueous solutions are preferably approximately isotonic, and its tonicity may be adjusted with agents such as sodium tartrate, sodium chloride, propylene glycol, and sodium phosphate. Additionally, auxiliary substances required to approximate physiological conditions, including pH adjusting and buffering agents, tonicity adjusting agents, wetting or emulsifying agents, pH buffering substances, and the like, including sodium acetate, sodium lactate, sodium chloride, potassium chloride, calcium chloride, sorbitan monolaurate, triethanolamine oleate, etc. may be included with the vehicles described herein.

These compositions may be sterilized by conventional sterilization techniques, or may be sterile filtered. The resulting aqueous solutions may be packaged for use as is, or lyophilized, the lyophilized preparation being combined with a sterile solution prior to administration. The preparation of such pharmaceutical compositions is within the ordinary skill in the art, and may be guided by standard reference books such as Remington's Pharmaceutical Science, supra.

Compositions may be formulated in a solid or liquid form for oral delivery. For solid compositions, nontoxic and/or pharmaceutically acceptable solid protein carriers may include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition may be formed by incorporating any of the normally employed excipients, including those protein carriers previously listed, and a unit dosage of an active ingredient, that is, one or more active agents disclosed herein.

The concentration of the active agents in the pharmaceutical compositions can vary widely, i.e., from less than about 0.1%, usually at or at least about 0.1% to as much as 20% to 50% or more by weight, and will be selected primarily by fluid volumes, viscosities, etc., and in accordance with the particular mode of administration selected. A human unit dose form of the active agents is typically included in a pharmaceutical composition that comprises a human unit dose of an acceptable protein carrier, preferably an aqueous protein carrier, and is administered in a volume of fluid that is known by those of skill in the art to be used for administration of such compositions to humans, and is adjusted according to commonly understood principles for a particular subject to be treated. In specific embodiments, a unit dosage of active agent is provided in a suitable amount of an aqueous solution, such as 0.1-3 mL, preferably 0.2-2 mL.

Such pharmaceutical compositions can be administered to subjects by a variety of mucosal administration modes, including by oral, rectal, intranasal, intrapulmonary, or transdermal delivery, or by topical delivery to other surfaces. Optionally, the active agents can be administered by non-mucosal routes, including by intramuscular, subcutaneous, intravenous, intra-atrial, intra-articular, intraperitoneal, or parenteral routes. Alternatively, the active agents can be administered ex vivo by direct exposure to cells, tissues or organs originating from a subject. In specific embodiments, the route of administration includes parenteral administration (e.g., intradermal, intramuscular or subcutaneous delivery), ocular administration, oral administration, intranasal administration, pulmonary administration, intravaginal administration, and intrarectal administration. Formulations of the pharmaceutical compositions may include liquid formulations for parenteral, subcutaneous, intradermal, intramuscular, intravenous, or ocular administration (e.g., injectable administration) such as sterile solutions, suspensions, or emulsions. Formulations of the pharmaceutical compositions also may include liquid formulations (e.g., topical formulations or ingestible formulations) for ocular, oral, nasal, anal, and vaginal administration, including solutions, suspensions, syrups or elixirs. The pharmaceutical compositions may be lyophilized prior to delivery and reconstituted prior to administration.

The pharmaceutical compositions of the disclosure typically are sterile and stable under conditions of manufacture, storage and use. Sterile solutions can be prepared by incorporating the conjugate in the required amount in an appropriate solvent with one or a combination of ingredients enumerated herein, as required, followed by filtered sterilization. Generally, dispersions are prepared by incorporating the active agent and/or other biologically active agent into a sterile vehicle that contains a basic dispersion medium and the required other ingredients from those enumerated herein. In the case of sterile powders, methods of preparation include vacuum drying and freeze-drying which yields a powder of the conjugate plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

### 6. Methods of Treatment

The compositions of the present disclosure may be administered to a subject that is infected with a Neisseria organism, or at risk for developing an infection by Neisseria organism. In accordance with the various treatment methods of the present disclosure, the active agents can be delivered to a subject in a manner consistent with conventional methodologies associated with management of Neisseria infection for which treatment or prevention is sought. In accordance with the disclosure herein, a prophylactically or therapeutically effective amount of the active agent is administered to a subject in need of such treatment for a time and under conditions sufficient to prevent, inhibit, and/or ameliorate a Neisseria infection or one or more symptom(s) thereof. For example, non-limiting symptoms of N. gonorrhoeae infections include pain in the lower abdomen, pelvis, testicle, or vagina, painful urination, abnormal vaginal discharge, discharge from penis, or increased vaginal discharge, fever, frequent urge to urinate, irregular menstruation, pus, or sore throat. Representative examples of N. meningitidis infections include fever and chills, fatigue, vomiting, cold hands and feet, severe aches or pains in the muscles, joints, chest, or abdomen, rapid breathing diarrhea, and in later stages, dark purple rash.

When composition comprises a chimeric molecule of the present disclosure as an active agent, the treatment may be given in a single dose schedule, or preferably a multiple dose schedule in which a primary course of treatment may be with 1-10 separate doses, followed by other doses given at subsequent time intervals required to maintain and or reinforce the response, for example, at 1-4 months for a second dose, and if needed, a subsequent dose(s) after several months. Examples of suitable treatment schedules include: (i) 0, 1 month and 6 months, (ii) 0, 7 days and 1 month, (iii) 0 and 1 month, (iv) 0 and 6 months, or other schedules sufficient to elicit the desired responses expected to reduce disease symptoms, or reduce severity of disease.

The amounts effective for inducing an immune response or providing protective immunity will depend on a variety of factors, including the oligosaccharide antigen component of the chimeric molecule, the associated carrier, inclusion and nature of any adjuvant(s), the manner of administration, the weight and general state of health of the patient, and the judgment of the prescribing physician. By way of example, the amounts may generally range for the initial immunization (that is for a prophylactic administration) from about 1.0 μg to about 5,000 μg of oligosaccharide antigen for a 70 kg patient, (e.g., 1.0 μg, 2.0 μg, 2.5 μg, 3.0 μg, 3.5 μg, 4.0 μg, 4.5 μg, 5.0 μg, 7.5 μg, 10 μg, 12.5 μg, 15 μg, 17.5 μg, 20 μg, 25 μg, 30 μg, 35 μg, 40 μg, 45 μg, 50 μg, 75 μg, 100 μg, 250 μg, 500 μg, 750 μg, 1,000 μg, 1,500 μg, 2,000 μg, 2,500 μg, 3,000 μg, 3,500 μg, 4,000 μg, 4,500 μg or 5,000 μg). The actual dose administered to a subject is often determined according to an appropriate amount per kg of the subject's body weight. For example, an effective amount may be about 0.1 μg to 5 μg/kg body weight.

A primary dose may optionally be followed by boosting dosages of from about 1.0 to about 1,000 of oligosaccharide antigen (e.g., 1.0 μg, 2.0 μg, 2.5 μg, 3.0 μg, 3.5 μg, 4.0 μg, 4.5 μg, 5.0 μg, 7.5 μg, 10 μg, 12.5 μg, 15 μg, 17.5 μg, 20 μg, 25 μg, 30 μg, 35 μg, 40 μg, 45 μg, 50 μg, 75 μg, 100 μg, 250 μg, 500 μg, 750 μg, 1,000 μg, 1,500 μg, 2,000 μg, 2,500 μg, 3,000 μg, 3,500 μg, 4,000 μg, 4,500 μg or 5,000 μg) pursuant to a boosting regimen over weeks to months depending upon the patient's response and condition by measuring specific antibody titers and/or T cell activity in the patient's blood.

The active agents may be optionally administered in combination with ancillary or adjunctive pharmaceutically active substances. For example, the active agents may be administered in combination with antimicrobials useful for treating Neisseria infections, representative examples of which include antibiotic(s) such as: Aminoglycosides (e.g., amikacin, gentamicin, kanamycin, neomycin, netilmicin, streptomycin, tobramycin and paromomycin); Ansamycins (e.g., geldanamycin and herbimycin); Carbacephems (e.g., loracarbef); Carbapenems (e.g., ertapenem, doripenem, imipenem/cilastatin and meropenem); Cephalosporins (first generation), including e.g., cefadroxil, cefazolin, cefalotin/cefalothin and cephalexin); Cephalosporins (second generation), including e.g., cefaclor, cefamandole, cefoxitin, cefprozil and cefuroxime; Cephalosporins (third generation), including e.g., cefixime, cefdinir, cefditoren, cefoperazone, cefotaxime, cefpodoxime, ceftazidime, ceftibuten, ceftizoxime, ceftriaxone and cefdinir; Cephalosporins (fourth generation), including e.g., cefepime; Glycopeptides (e.g., vancomycin and teicoplanin); Macrolides (e.g., azithromycin, clarithromycin, dirithromycin, erythromycin, roxithromycin, troleandomycin, telithromycin and spectinomycin); Monobactams (e.g., aztreonam); Penicillins (e.g., amoxicillin, ampicillin, azlocillin, carbenicillin, cloxacillin, dicloxacillin, flucloxacillin, mezlocillin, nafcillin, penicillin, piperacillin and ticarcillin); Polypeptides (e.g., bacitracin, polymixin B and colistin); Quinolones (e.g., ciprofloxacin, enoxacin, gatifloxacin, levofloxacin, lomefloxacin, moxifloxacin, norfloxacin, ofloxacin and trovafloxacin); Sulfonamides (e.g., mafenide, prontosil, sulfacetamide, sulfamethizole, sulfanilimide, sulfasalazine, sulfisoxazole, trimethoprim, trimethoprim-sulfamethoxazole (co-trimoxazole, TMP-SMX)); Tetracyclines (e.g., demeclocycline, doxycycline, minocycline, oxytetracycline and tetracycline); Aminocoumarins (e.g., novobiocin, albamycin, coumermycin and clorobiocin); Oxazolidinones (e.g., linezolid and AZD2563); Lipopeptides (e.g., daptomycin); Streptogramins (e.g., quinupristin/dalfopristin); Glycylcyclines (e.g., tigecycline); Lantibiotics (e.g., Type A Lantibiotics (such as nisin, subtilin, epidermin, mutacin II, mutacin I & III) and Type B Lantibiotics (such as mersacidin, actagardine and cinnamycin). Other suitable antibiotics useful as adjunctive agents include one or more antibiotic(s) selected from: arsphenamine, chloramphenicol, clindamycin, lincoamycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampin/rifampicin and tinidazole.

Thus, the active agents disclosed herein may be used in combination with one or more antibiotics selected from: penicillin, cloxacillin, dicloxacillin, methicillin, nafcillin, oxacillin, ampicillin, amoxicillin, bacampicillin, capreomycin, cycloserine, azlocillin, carbenicillin, mezlocillin, piperacillin, ticarcillin, azithromycin, clarithromycin, clindamycin, erythromycin, lincomycin, demeclocycline, doxycycline, ethambutol, ethionamide, minocycline, oxytetracycline, tetracycline, quinolone, cinoxacin, nalidixic acid, fluoroquinolones (e.g., levofloxacin, moxafloxacin and gatifloxacin, ciprofloxacin, enoxacin, grepafloxacin), kanamycin, levofloxacin, lomefloxacin, norfloxacin, ofloxacin, p-aminosalicylic acid, sparfloxacin, trovafloxacin, bacitracin, colistin, polymyxin B, sulfonamide, trimethoprim-sulfamethoxazole, co-amoxyclav, cephalothin, cefuroxime, ceftriaxone, vancomycin, gentamicin, amikacin, metronidazole, chloramphenicol, streptomycin, nitrofurantoin, co-trimoxazole, rifamycin and derivatives thereof (e.g., rifampicin, rifabutin and rifapentine), isoniazid, pyrazinamide, kirromycin, thiostrepton, micrococcin, fusidic acid, thiolactomycin and fosmidomycin. Other suitable antibacterial adjunctive agents may be selected from those listed in the table below: DU-6859, Erythromycin stinoprate, Oritavancin, Telavancin, Dalbavancin, Ceftobiprole medocaril, Tebipenem pivoxil, Iclaprim, OPT-80, Ceftaroline fosamil, RX-3341, Cethromycin, TD-1792, EDP-420, RX-1741, MK-2764, Nemonoxacin, Flopristin+Linopristin, Tomopenem, Ramoplanin, Linezolid, Cefditoren pivoxil, Ertapenem, Gemifloxacin, Daptomycin, Telithromycin, and Tigecyline.

In some embodiments, a chimeric molecule of the present disclosure is used in combination with one or more immunogens derived from Neisseria antigens representative examples of which include AniA, BamA, CssA, CssB, CssC, CtrA, CtrB, CtrC, CtrD, CtrE, CtrF, ExbB, GNA1030, GNA1162, GNA1220, GNA1870 (fHbp), GNA1946, GNA2001, GNA2091, GNA2132 (NHBA), GNA33, GNA992, LctP, LpdA, LptD, MsrAB, NadA, NGO2054, NGO2139 (MetQ), NspA, OpaD, OpcA, PilC1, PilQ, PorA P1, TamA, TBP2, and TbpA. In specific embodiments, the immunogens are selected from AniA, MsrAB, MetQ and NhbA.

Typical subjects intended for treatment with the compositions and methods of the present disclosure include humans, as well as non-human primates and other animals. To identify subjects for prophylaxis or treatment according to the methods of the disclosure, accepted screening methods are employed to determine risk factors associated with a targeted or suspected disease of condition (e.g., coughing disease) as discussed herein, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods, which are available and well known in the art to detect and/or characterize disease-associated markers. These and other routine methods allow the clinician to select patients in need of therapy using the methods and pharmaceutical compositions of the disclosure. In accordance with these methods and principles, a conjugate and/or other biologically active agent can be administered according to the teachings herein as an independent prophylaxis or treatment program, or as a follow-up, adjunct or coordinate treatment regimen to other treatments, including surgery, vaccination, immunotherapy, hormone treatment, cell, tissue, or organ transplants, and the like.

The administration of the active agents of the disclosure can be for either prophylactic or therapeutic purpose. When provided prophylactically, the active agent is provided in advance of any symptom. The prophylactic administration of the active agent serves to prevent or ameliorate any subsequent infection. When provided therapeutically, the conjugate is provided at (or shortly after) the onset of a symptom of disease or infection. An active agent of the disclosure can thus be provided prior to an anticipated exposure to a Neisseria organism so as to attenuate the anticipated severity, duration or extent of an infection and/or associated disease symptoms, after exposure or suspected exposure to the bacteria, or after the actual initiation of an infection.

The methods of using the active agents, and the related compositions and methods of the disclosure, are useful in increasing resistance to Neisseria organisms, and/or preventing, ameliorating, and/or treating infection and disease caused by Neisseria organisms, in animal hosts, and other, in vitro applications. Compositions comprising chimeric molecules of the present disclosure can be used for active immunization for prevention of infection, and for preparation of immune antibodies.

### 7. Kits

The instant disclosure also includes kits for practicing the methods of the disclosure. These reagents and kits may vary and will typically contain any one or more of the active agents or compositions disclosed herein, optionally including instructions or means for administering the same for use in the prevention and treatment of Neisseria infections in vertebrate subjects. Kits for diagnostic use are also provided. In some embodiments, these kits include a container or formulation that contains one or more of the chimeric molecules described herein. In one example, this component is formulated in a pharmaceutical preparation for delivery to a subject. A chimeric molecule of the present disclosure is optionally contained in a bulk dispensing container or unit or multi-unit dosage form. Optional dispensing means can be provided. Packaging materials optionally include a label or instruction indicating for what treatment purposes and/or in what manner the active agent packaged therewith can be used. The pharmaceutical compositions or components may be provided in any suitable form (e.g., liquid form or lyophilized form). Kits further may include solvents for resuspending or dissolving a lyophilized active agent.

In order that the disclosure may be readily understood and put into practical effect, particular preferred embodiments will now be described by way of the following non-limiting examples.

### EXAMPLES

### Example 1

### Demonstration that KDO is Surface Expressed in N. gonorrhoeae

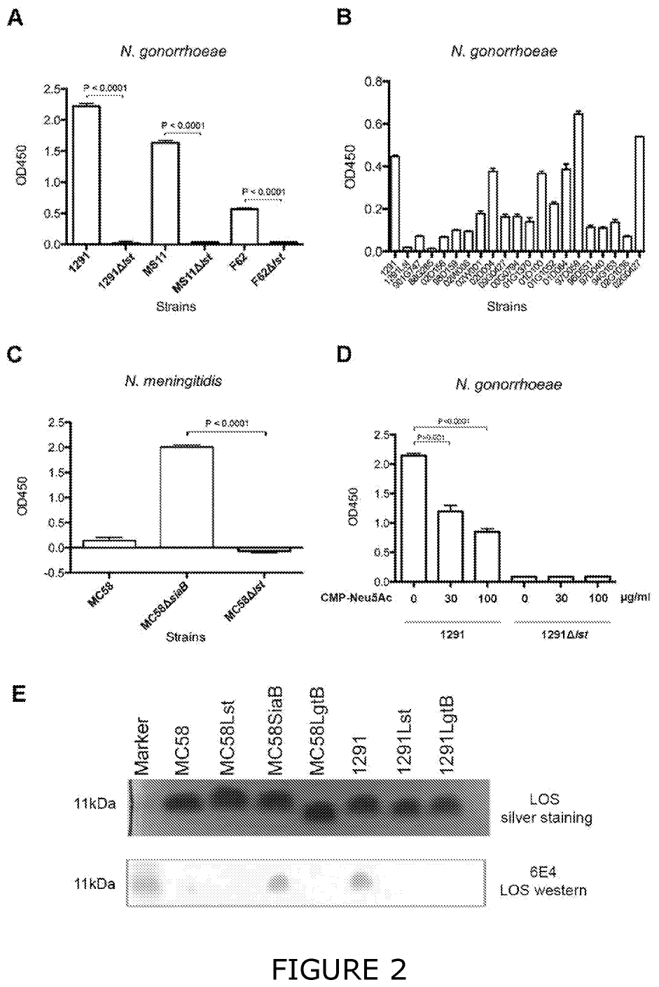

Blast analysis indicated that N. gonorrhoeae sialyltransferase Lst belongs to the glycosyltransferase superfamily 52 and is homologous to the sialyltransferase LsgB in NTHi (27% identity at the amino acid level, e value=6e-31). To determine whether N. gonorrhoeae can transfer KDO as the terminal monosaccharide of LOS, similar to NTHi, whole cell ELISA was performed with the LOS terminal KDO specific monoclonal antibody (MAb) 6E4 on three well characterized N. gonorrhoeae strains, 1291, MS11 and F62 and their lst knockout mutants. All three wild-type strains were KDO positive, while all lst mutants of these strains did not react with MAb 6E4 (FIG. 2A), indicating that Lst is required for KDO transfer to LOS. Consistent with previous study, 6E4 cannot detect the basal KDO residue that is common to all LOS structure. Further analysis detected KDO (FIG. 26) in 19 of 20 gonococcal clinical isolates from patients with mucosal or disseminated gonococcal infections. Variable presentation of KDO on gonococcal LOS may be due to the absence of a terminal galactose acceptor, if LgtA is switched off, or via competition if another glycosyltransferase, LgtD, is phase varied “on” and transfers GalNAc in competition with Lst/CMP-KDO for the terminal galactose acceptor. The transfer of KDO to LOS was also investigated in N. meningitidis, which unlike N. gonorrhoeae, can synthesize CMP-Neu5Ac via SiaB. The ELISA results in FIG. 2C show the absence of KDO in meningococcal strain MC58 wild-type and lst mutant. However, a MC58 siaB mutant, which cannot synthesize CMP-Neu5Ac, was recognized by MAb 6E4, indicating the presence of KDO. These data indicate that there is competition between the CMP-Neu5Ac and CMP-KDO substrates for Lst mediated transfer of the terminal sugar to LOS, and that a mutation in N. meningitidis that ablates CMP-Neu5Ac synthesis is sufficient to permit KDO addition to LOS. To define the terminal KDO structure on the LOS, MAb 6E4 Western blot with LOS purified from MC58, 1291 and their LOS glycosyltransferase (lst and lgtB) mutant strains was performed. LgtB is a β1,4-galactosyltransferase that transfers CMP-Neu5Ac and CMP-KDO receptor galactose (Gal) to the N-acetylglucosamine (GlcNAc). As shown in FIG. 2E, the purified LOS from the 1291 wild type and the MC58 siaB mutant was 6E4 positive, while the 1291 and MC58 lst and lgtB mutants were all 6E4 negative. LOS from MC58 wild-type was slightly 6E4 positive, indicating the presence of a small amount of KDO incorporated terminal on LOS.

To determine whether the incremental addition of CMP-Neu5Ac to the growth media can complete with CMP-KDO for Lst addition to LOS, and thereby reduce binding of MAb 6E4, N. gonorrhoeae strain 1291 was grown in 0 μg/mL, 30 μg/mL and 100 μg/mL CMP-Neu5Ac and whole cell ELISA was performed. The results presented in FIG. 2D show that dose dependent inhibition of 6E4 MAb binding to whole cells begins at 30 μg/mL CMP-Neu5Ac and is increased at 100 μg/mL. These data suggest that CMP-Neu5Ac taken up from the media competes with endogenous CMP-KDO as a substrate for Lst activity.

### Example 2

### The KDO Antigen is Expressed In Vivo

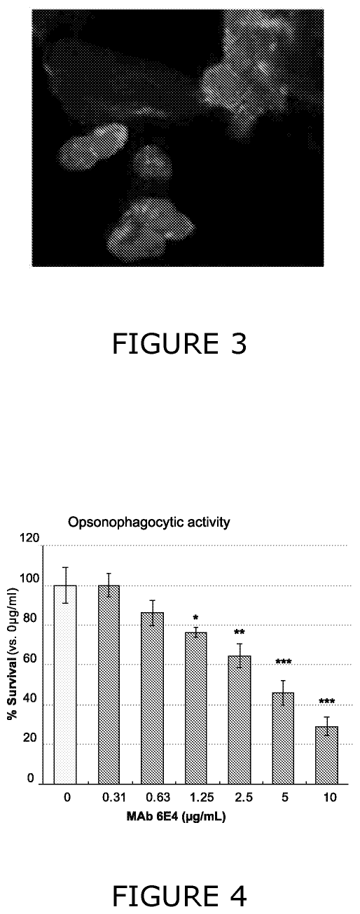

To examine if N. gonorrhoeae can incorporate KDO as the terminal glycan of LOS during in vivo infection, a confocal microscopy study was conducted using MAb 6E4 on the cervical swab from a patient with a documented N. gonorrhoeae infection. FIG. 3 demonstrates that in vivo, the terminal KDO epitope is present in N. gonorrhoeae on cervical epithelial cells. This suggests that the free CMP-Neu5Ac in the cervical environment was not sufficient to inhibit Lst incorporating KDO to LOS.

### Example 3

### Mab 6E4 Mediates Opsonophagocytic Killing Via Targeting the KDO Epitope

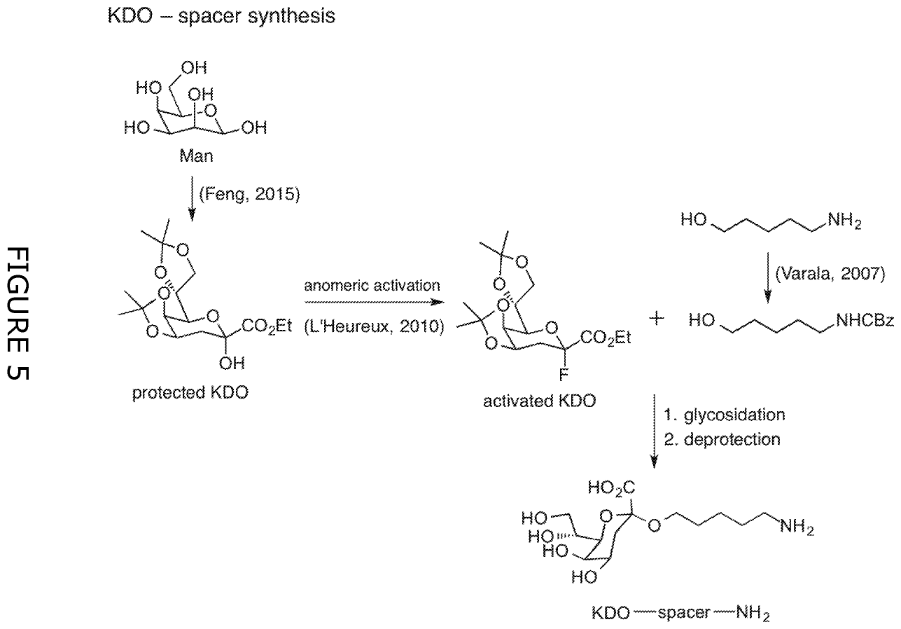

To examine if the surface expressed KDO in N. gonorrhoeae could be a vaccine target, MAb 6E4 was used in opsonophagocytic killing assays against N. gonorrhoeae strain 1291. Dose-dependent opsonophagocytic killing was observed from 0.65-10 μg/mL of MAb 6E4, with 5 μg/mL of MAb 6E4 eliciting 54% killing and 10 μg/mL eliciting 71% killing relative to no treatment control (FIG. 4). These data suggest that antibodies targeting KDO can mediate bacterial killing via opsonophagocytic activity and that LOS terminal KDO epitope in N. gonorrhoeae is a potential vaccine target.

### Example 4

### KDO Monosaccharide Presented on a Carrier Protein CRM-197 Elicits an Immune Response in Mice

KDO—Spacer Synthesis

Commercially available mannose is converted into the protected KDO derivative in 2 steps, following the method described by the Feng group (Feng et al. 2015, Org Lett. 17(10):2388-91). The protected KDO is then activated to the glycosyl fluoride following the procedure of L'Heureux et al. (2010, J Org Chem. 75(10):3401-11). Commercially available 5-amino-1-pentanol has the amine group protected following the method described by Varala et al. (2007, J Iran Chem Soc. 4: 370-374). Glycosidation between the activated KDO and the spacer is carried out using a procedure described by Boltje et al. (2012, J Am Chem Soc. 134, 14255-14262). Deprotection then provides the KDO-spacer compound for conjugation. FIG. 5 shows steps of KDO-spacer chemical synthesis.

KDO Spacer Characterization

All small molecule chemical entities synthesized in this work are characterized using standard spectroscopic techniques, notably Nuclear Magnetic Resonance (NMR, both 1H and 13C), mass spectrometry, and infrared spectroscopy. Proof that the correct glycosidic linkage orientation has been formed when KDO is joined with the spacer is determined using NMR spectroscopy, particularly the chemical shift of the anomeric carbon and the protons attached to carbon-3 as per Kong, et al. (Kong et al. 2016, Nat Chem. 8:242-249).

KDO Conjugation to CRM197 and BSA (or Other Carriers)

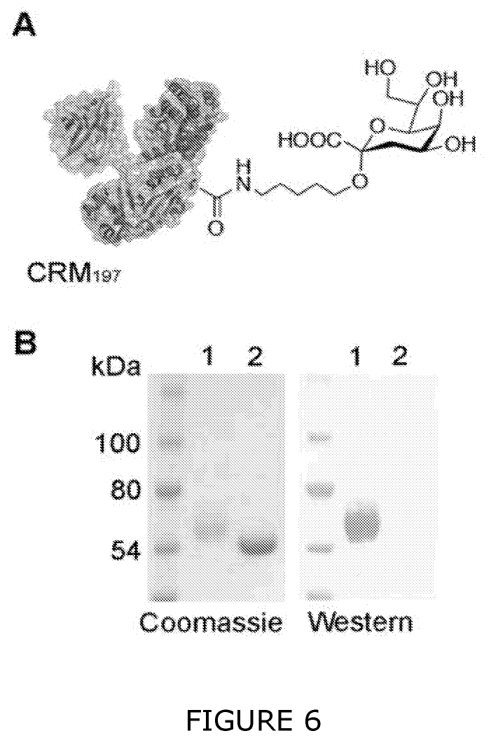

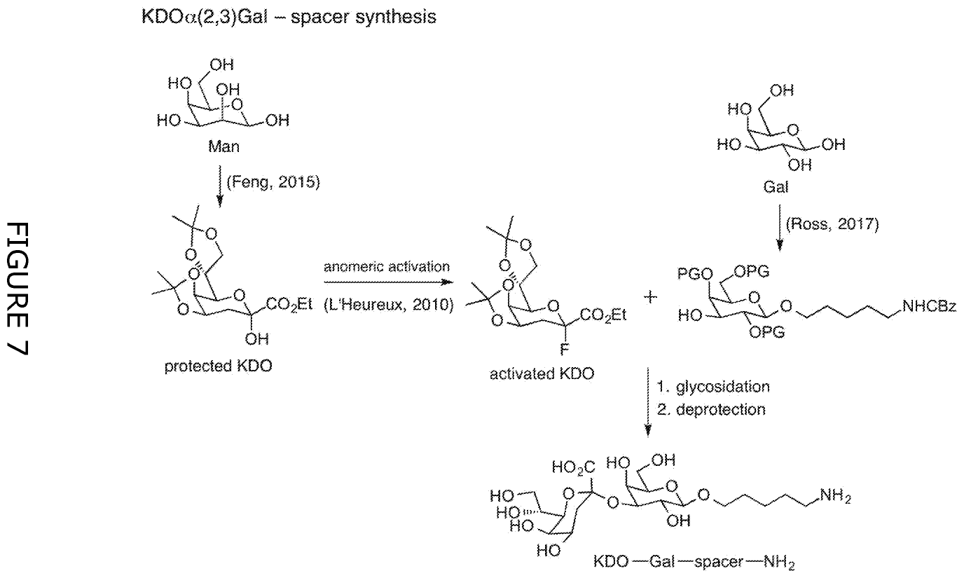

The chemical structure of KDO conjugation to CRM197 is shown in FIG. 6A. CRM197 (or BSA and other carriers) in 0.3 M NaHCO3(1 mg/mL) was added to NCS KDO-linked (200 molar equiv.) in 0.3 M Na₂CO₃ pH 9 to a final volume of 1 mL. The resultant mixture was gently shaken for 18 hrs. at 20° C. SDS-PAGE and Western Blot were used to analyze reaction product. In the EDC-NHS reaction, CRM197 and NH₂ linked KDO were incubated in EDC (25 mM) and NHS (25 mM) (GE, EDC-NHS coupling kit) for 30 min at room temperature. The resultant conjugation reactions were stopped by passing through the desalting columns (Bio-Spin™, Bio-Rad) to remove excess un-conjugated glycans and buffer exchanged to PBS. SDS-PAGE western blot of 6E4 as shown in FIG. 6B, and MS were used to analyze and confirm the glyco-conjugated CRM197.

Murine Antigenicity Studies

The analysis of murine antigenicity study was performed as previously published (Jen et al, 2015, Front Immunol. 10:137). To evaluate the immunogenicity of the KDO monosaccharide based glycoconjugate construct, 10 mice were each immunized with 100 μg of KDO glycoconjugate or the native carrier protein (CRM197) in a 1:1 formation with Alhydrogel™ adjuvant. Two booster injections were administered at days 21 and 28 after initial injection, with a terminal bleed scheduled at day 42. All animals remained healthy for the duration of the study, and no adverse reactions were observed. Terminal bleed sera were analyzed for an anti-KDO immune response via ELISA assay.

Analysis of Immune Response

To investigate the immunogenicity of CRM-KDO, ten mice were immunized with CRM-KDO with Alhydrogel™ adjuvant. The sera were assessed by ELISA. Wells of plates were coated with 100 ng of BSA-KDO glycoconjugate protein in 100 μL of coating buffer (0.5M carbonate/bicarbonate buffer, pH 9.6) for 1 hr. at room temperature. All ELISAs were performed with mouse pre-immune or CRM-KDO immunized sera, and secondary antibody, polyclonal anti-mouse Ig HRP (Dako). The substrate TMB (3,3′, 5,5;-tetramethylbenzidine) solution (Thermofisher Scientific) was used as per manufacturer's instruction. Equal amount of 1 N hydrochloric acid was added to stop the reaction. Absorbance was read in a TECAN Model Infinite 200 Pro plate reader at 450 nm. ELISA results with BSA-KDO as the coating antigen indicate KDO specific immune response in mice immunized with CRM-KDO, with a titer of >25,000 as shown in Table 1.

TABLE 1
ELISA titer data for pooled mice sera
immunized with KDO-CRM197 conjugate.

| | | KDO-CRM197 | | CRM197 |
|---|---|---|---|---|
| Sera | KDO-CRM197 | Pre-immune | CRM197 | Pre-immune |
| ELISA titer | >25,000 | 200 | 200 | 200 |

### Example 5

### Synthesis of KDO Antigens (I) Chemical Synthesis of KDO Di- and Tri-Saccharide Antigens

KDOα(2,3)Gal-Spacer Synthesis

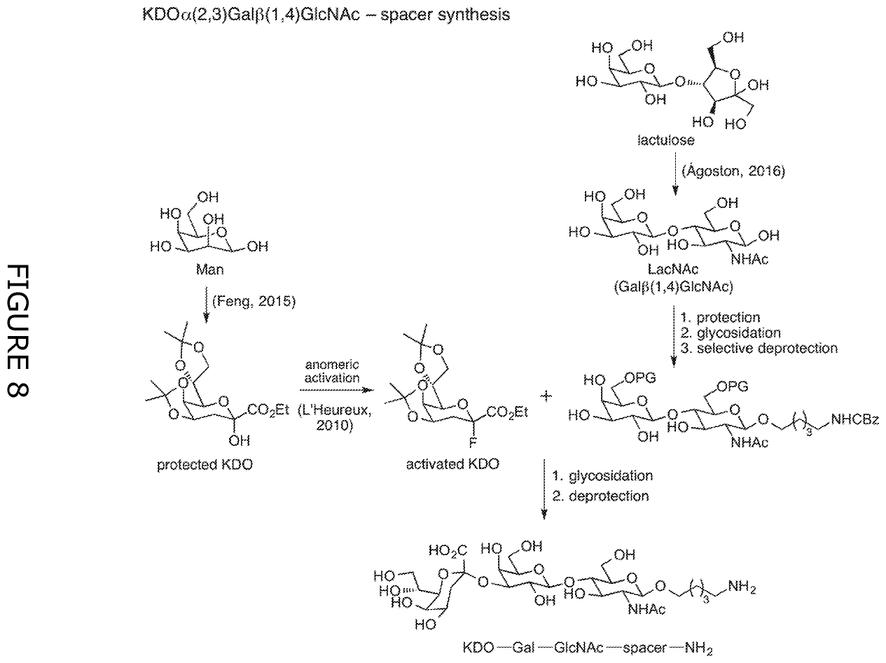

Commercially available mannose is converted into the protected KDO derivative in 2 steps, following the method described by the Feng group (Feng et al. 2015, supra). The protected KDO is then activated to the glycosyl fluoride following the procedure of L'Heureux et al. (2010, supra). Commercially available galactose is converted into the Gal-spacer compound using standard carbohydrate chemistry developed in house. Glycosidation between the activated KDO and the Gal-spacer is accomplished using a method adapted from the literature (Kong et al. 2016, supra). Deprotection then provides the KDOα(2,3)Gal-spacer compound for conjugation. FIG. 7 shows steps of KDOα(2,3)Gal-spacer chemical synthesis.

KDOα(2,3)Galβ(1,4)GlcNAc—Spacer Synthesis

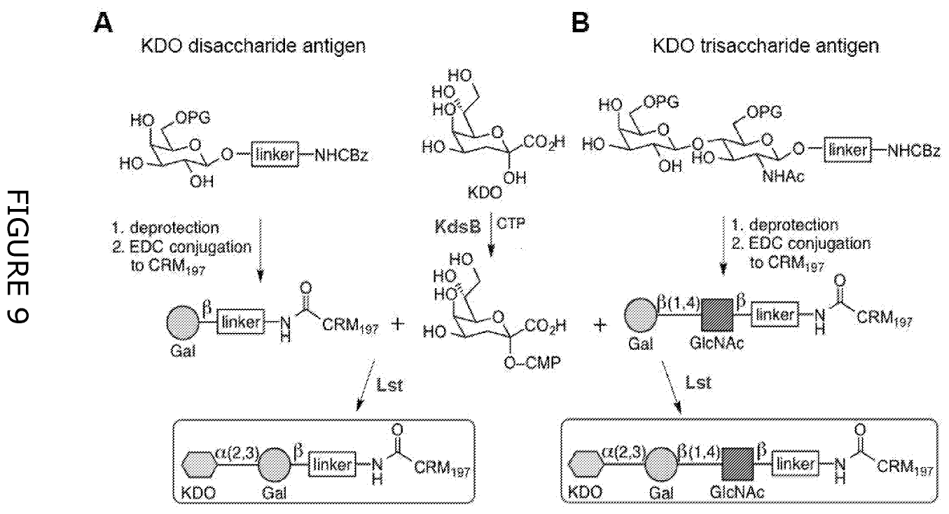

Commercially available mannose is converted into the protected KDO derivative in 2 steps, following the method described by the Feng group (Feng et al. 2015, supra). The protected KDO is then activated to the glycosyl fluoride following the procedure of L'Heureux, et al. (2010, supra). Commercially available lactulose is converted into LacNAc (Galβ(1,4)GlcNAc) using the method described by Agoston et al. (2016, Tet Lett. 57: 2595-2597). Through a series of standard carbohydrate protecting group manipulations developed in house, LacNAc is converted into the Galβ(1,4)GlcNAc-spacer compound. Glycosidation between the activated KDO and the Galβ(1,4)GlcNAc-spacer is accomplished using a method adapted from the literature (Kong et al. 2016, supra). Deprotection then provides the KDOα(2,3)Galβ(1,4)GlcNAc-spacer compound for conjugation. FIG. 8 shows steps of KDOα(2,3)Galβ(1,4)GlcNAc-spacer chemical synthesis.

Di- and Tri-Saccharide KDO-Spacer Characterization (i.e., Post-Synthesis)

All small molecule chemical entities synthesized in this work are characterized using standard spectroscopic techniques, notably Nuclear Magnetic Resonance (NMR, both 1H and 13C), mass spectrometry, and infrared spectroscopy. Proof that the correct glycosidic linkage orientation has been formed when KDO is joined with the Gal is determined using NMR spectroscopy, particularly the chemical shift of the anomeric carbon and the protons attached to carbon-3 as per Kong, et al. (2016, supra).

Di- and Tri-Saccharide KDO Antigen Conjugation to CRM197 and BSA

CRM197 (alternatively, BSA or other carriers) in 0.3 M NaHCO₃ (1 mg/mL) was added to NCS KDO-linked (200 molar equiv.) in 0.3 M Na₂CO₃ pH 9 to a final volume of 1 mL. The resultant mixture was gently shaken for 18 hrs. at 20° C. SDS-PAGE and western blot were used to analyze reaction product. In the EDC-NHS reaction, CRM197 and NH₂ linked KDO were incubated in EDC (25 mM) and NHS (25 mM) (GE, EDC-NHS coupling kit) for 30 min at room temperature. The resultant conjugation reactions were stopped by passing through the desalting columns (Bio-Spin™, Bio-Rad) to remove excess un-conjugated glycans and buffer exchanged to PBS. SDS-PAGE western blot of 6E4, and MS were used to analyze and confirm the glyco-conjugated CRM197.

Murine Antigenicity Studies

To evaluate the immunogenicity of the KDO di- and tri-saccharide based glycoconjugate construct, 10 mice will be each immunized with 25, 50 or 100 μg of KDO glycoconjugate or the native carrier protein (CRM197) in a 1:1 formation with Alhydrogel™ adjuvant. Two booster injections will be administered at days 21 and 28 after initial injection, with a terminal bleed scheduled at day 42. All animals remained healthy for the duration of the study, and no adverse reactions were observed. Terminal bleed sera will be analyzed for an anti-KDO immune response via ELISA assay.

Analysis of Immune Response

The analysis of immune response will be performed as previously published (Jen et al, 2015, supra). ELISA will be used to determine antibody titers and isotypes for sera raised, using plates coated with either the BSA-KDO antigens or with N. gonorrhoeae whole cells. Wells of plates will be coated with 100 ng of BSA-KDO glycoconjugate and BSA protein in 100 μL of coating buffer (0.5M carbonate/bicarbonate buffer, pH 9.6) for 1 hr. at room temperature or overnight at 4° C. to test KDO specific immune response. All ELISAs will be performed with mouse pre-immune or CRM-KDO immunized sera, and secondary antibody, polyclonal anti-mouse Ig HRP (Dako) or IgG1, IgG2a, IgG2b, IgG3, or IgM HRP (Thermofisher Scientific). The substrate TMB (3,3′, 5,5 tetramethylbenzidine) solution (Thermofisher Scientific) will be used as per manufacturer's instruction. Equal amount of 1 N hydrochloric acid will be added to stop the reaction. Absorbance will be read in a TECAN Model Infinite 200 Pro plate reader at 450 nm.

### Example 6

### Synthesis of KDO Antigens (II) Chemoenzymatic Synthesis of KDO Di- and Tri-Saccharide Antigens

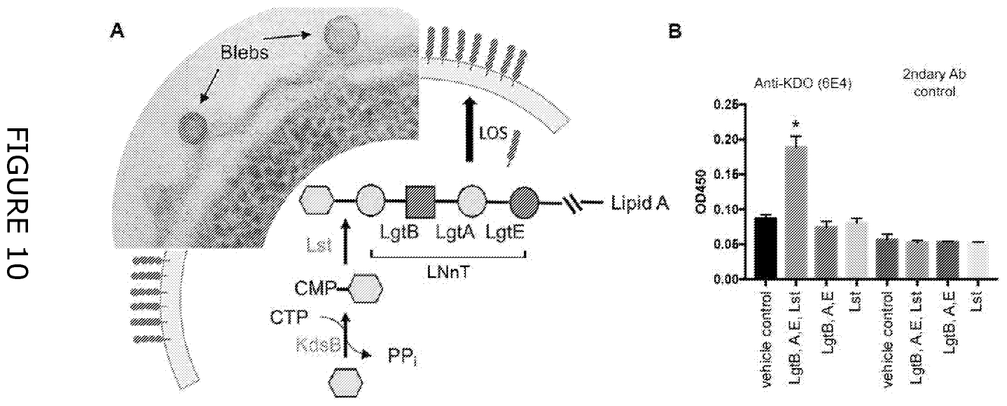

As shown if FIG. 9, the Gal derivative or the LacNAc derivative from EXAMPLE 5 are deprotected with the free NCS or NH₂ at the end of the linker that enables EDC-NHS coupling with CRM197. Having successfully conjugated the Gal and LacNAc compounds via a linker to CRM197, the next phase of the chemoenzymatic approach involves using enzyme KdsB (CMP-KDO synthetase from E. coli) based on (Sugai et al. 1995, Bioorg Med Chem. 3(3):313-20) to activate KDO in situ to the CMP-KDO derivative, which can then be transferred by the glycosyltransferase Lst (sialyltransferase from Neisseria) base on (Gilbert et al. 1996, J Biol Chem. 271:28271-6) to the sugar ligand already conjugated to CRM197.

### Example 7

### Synthesis of KDO Antigens (III) In Vivo Synthesis of KDO Antigens

### Overexpress KDO in N. gonorrhoeae OMVs as a Vaccine Candidate

As shown in FIG. 10A, the CMP-KDO synthetase (KdsB) and CMP-KDO transferase (Lst) will be transferred to the N. gonorrhoeae chromosome on an IPTG inducible expression construct. The level of terminal KDO expression on LOS will be determined by ELISA using MAb 6E4 and MS. Naturally-secreted outer membrane vesicles (OMVs) will be isolated/purified by two previously described methods: natively purified by ultracentrifugation (Semchenko et al. 2017, Infect Immun. 85(2)) and deoxycholate (DOC) detergent extracted (Fredriksen et al. 1991, NIPH Ann. 14(2):67-79). These approaches result in different OMV composition and yield, and both will be investigated. Native N. gonorrhoeae OMVs will be isolated from a 6-hour inoculated culture (OD600˜0.8) by brief centrifugation (5,000×g) and subsequent filtration of the supernatant (0.22 μm filter). The filtrate is then centrifuged (100,000×g, 1 hr., 4° C.), the pellet containing OMVs washed three times with PBS, and then solubilized in PBS containing 0.2% SDS. For detergent extraction, a 6-hour inoculated culture is incubated in 0.1 M Tris-HCl, pH 8.6, containing 10 mM EDTA and 0.5% DOC for 30 min at room temperature, then centrifuged (20,000×g; 30 min; 4° C.). The supernatant is ultracentrifuged (125,000×g; 2 hrs.; 4° C.) and the OMV pellet is resuspended in 50 mM Tris-HCl, pH 8.6, 2 mM EDTA, 1.2% DOC, 20% sucrose for second ultracentrifugation step. OMVs will then be homogenized in 30% sucrose. All purified OMVs will be analyzed by SDS-PAGE and protein concentration measured using the BCA protein assay.

Groups of 10 BALB/c mice will be immunized with 5 μg of OMVs with Alhydrogel™ (aluminum hydroxide, InvivoGen) on days 0, 21, and 28. Terminal bleeds will be collected on day 42, as per standard procedures. Control mice will be immunized with 5 μg native OMV, or a PBS sham, with Alhydrogel™. A pre-bleed of each mouse will be collected 4 days before immunization. We will then use ELISA to determine antibody titers and isotypes for sera raised, using plates coated with either the immunizing antigens or N. gonorrhoeae whole cells.

### Overexpress KDO in an Engineered, Reduced Toxicity, Controlled Blebbing Escherichia coli Strain to Assess KDO-OMVs as Vaccine Candidates

An E. coli endotoxin reduced and OMV upregulated strain (C600ΔwaaOBΔlppΔmsbBattλ::ParaB-lppΔompA:pGb3) has been generated as a strain to express heterologous oligosaccharides and may be used to deliver carbohydrate vaccine antigens (Paton et al. 2015 Infect Immun. 83(9):3526-33). To enable expression of the terminal KDO tetrasaccharide in E. coli, the plasmids pLNnT (a LgtE, LgtA and LgtB expression construct) and pBAD Lst will be transformed into this E. coli strain. As shown in FIG. 10A, LgtE, LgtA, LgtB and Lst, encode a β-1,4-galactosyltransferase, a β-1,3-N-acetylglucosaminyltransferase, a β-1,4-galactosyltransferase and sialyltransferase with distinct acceptor specificity, respectively. Whole cell 6E4 ELISA as shown in FIG. 10B and MS were used to confirm KDO expression on LOS in the E. coli blebbing strain. Blebbing of this strain will be induced by supplementing 0.2% glucose in growing culture, and the OMVs will be isolated by centrifugation (3,900×g at 4° C. for 10 min, then again for 15 min to remove whole cells). The resulting supernatant will be centrifuged at 150,000×g for 3 hours at 4° C. The pellet containing OMVs will be washed with PBS before a second centrifugation at 120,000 g for 30 min. at 4° C. The OMVs in the pellet will be resuspended in PBS for analysis by SDS-PAGE and BCA protein assay.

Murine antigenicity studies of purified OMVs will be performed as previously published (Jen et al, 2015, supra). Groups of 10 BALB/c mice will be immunized with 5 μg of OMVs with Alhydrogel™ (aluminum hydroxide, InvivoGen) on days 0, 21, and 28. Terminal bleeds are collected on day 42, as per standard procedures. Control mice will be immunized with native E. coli OMVs and a PBS sham with Alhydrogel™. A pre-bleed of each mouse will be collected 4 days before immunization. We will then use ELISA to determine antibody titers and isotypes for sera raised, using plates coated with either the immunizing antigens or with N. gonorrhoeae whole cells.

Alternately the same terminal KDO tetrasaccharide antigen shown in FIG. 10 can be generated in E. coli and then instead of OMVs, minicells or bacterial ghosts may be prepared to present the KDO carbohydrate vaccine antigen (Paton et al. 2015 Infect Immun. 83(9):3526-33). A further in vivo strategy for heterologous expression of the KDO antigen shown in FIG. 10 is to express appropriate glycosyltransferases to generate the desired KDO antigen that can be conjugated in vivo to a carrier protein using established approaches (Ravenscroft et al. 2019 Glycobiology. 29(9): 669-680).

### Example 8

### Human Serum Titer Against KDO Antigens

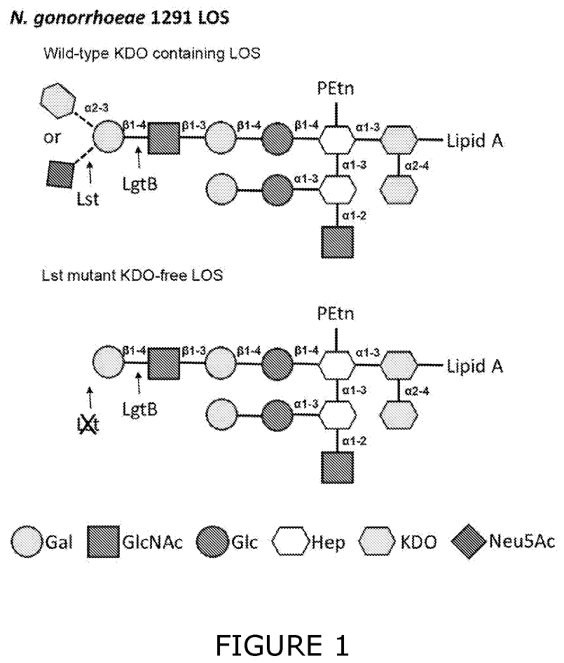

To evaluate if normal human sera (NHS) contain anti-KDO antibody, ELISAs of KDO antigens probing with NHS were performed. 100 ng of purified N. gonorrhoeae LOS with KDO (wild-type LOS, see structure in FIG. 1) and without KDO (Lst mutant LOS, see structure in FIG. 1) were used as the coating antigens on the ELISA plates. 10 individual NHS samples and commercially available pooled NHS from Sigma-Aldrich (catalogue #S1-M) were used to test if they contain antibody targeting KDO. KDO specific titer for each NHS sample was measured by subtracting the reading of LOS without KDO (Lst mutant LOS) from the reading of LOS with KDO (wild-type LOS). The endpoint was determined by the positive reading above the secondary antibody only control. ELISA results showed that human sera has a measurable anti-KDO titer as shown in Table 2. 6E4 is used as the positive control.

TABLE 2
ELISA titer data for normal human sera
containing KDO specific antibodies.

| Antibodies/sera | KDO specific titer |
|---|---|
| MAb 6E4 * | 1000 |
| NHS (sigma) | 4000 |
| serum 1 | 1000 |
| serum 2 | 400 |
| serum 3 | 1000 |
| serum 4 | 2000 |
| serum 5 | 2000 |
| serum 6 | 400 |
| serum 7 | 1000 |
| serum 8 | 400 |
| serum 9 | 2000 |
| serum 10 | 4000 |

* 6E4 is unpurified MAb culture supernatant.

### Materials and Methods

Bacterial Strains and Growth Conditions

N. gonorrhoeae strain 1291, 1291lst, MS11, MS11lst, F62, and F62lst (Mubaiwa et al. 2017, Sci Rep 7:5693) were grown on GC agar (Oxoid) or GC broth with 1% IsoVitaleX and N. meningitidis strain MC58, MC58lst (Mubaiwa et al. 2017, Sci Rep 7:5693) and MC58siaB (Chopra et al, submitted) were grown on brain-heart infusion (BHI) 1% agar and 10% Levinthal's Medium Base at 37° C. with 5% CO₂. Twenty clinical isolates (Power et al. 2007, Infect Immun 75:3202-4) of N. gonorrhoeae from disseminated or mucosal gonorrhoeae infection were also studied. In selected experiments, CMP-Neu5Ac at varying concentrations was added to the medium as indicated.

Lipooligosaccharides (LOS) Purification

Cells were grown on BHI or GC solid medium. Bacteria were collected by scraping and were suspended in a solution of 60 mM TRIS, 10 mM EDTA, and 2% (wt/vol) sodium dodecyl sulfate, pH 6.8. Proteinase K was added to a final concentration of 50 μg/ml and samples were incubated overnight at 37° C. Following three ethanol precipitations, samples were treated with DNase I and RNase A. Samples were then phenol extracted, ethanol precipitated three more times, and centrifuged at 120,000×g for 75 minutes. The pellets were resuspended in water, frozen, and then lyophilized.

Whole-Cell ELISA

Bacteria were grown on BHI or GC plates for 16 hr. Cells were harvested and resuspended in PBS at an OD 600 nm of 0.20. Microtiter plate wells were filled with 50 μL of the bacterial suspension and dried at room temperature overnight in the laminar flow cabinet. After the bacteria in the dried wells were heat-killed for 1 hr. in 56° C., the wells were washed, and ELISA was performed with MAb 6E4 at a dilution of 1:64. Secondary antibody (Dako P044701 polyclonal anti-mouse Ig HRP) was used at a dilution of 1:2,000. The substrate TMB (3,3′, 5,5;-tetramethylbenzidine) solution (Thermofisher Scientific) was used as per manufacturer's instruction. Equal amount of 1 N hydrochloric acid was added to stop the reaction. Absorbance was read in a TECAN Model Infinite 200 Pro plate reader at 450 nm.

Fluorescent Labeling of Clinical Samples

Paraformaldehyde fixed archival clinical specimens from cervical swabs obtained from patients presenting with gonorrhea were also dried onto glass microscope slides. These samples were incubated simultaneously with MAbs 6E4 (IgG) and 664 (IgM). Fluorescent label was provided by incubation with Goat anti-mouse IgG3/FITC to label MAb 6E4, plus Goat anti-mouse IgM/Texas Red for MAb 664 (both conjugates from Jackson ImmunoResearch). The samples were counter-stained with Draq5 (Cell Signaling Technologies) to label cell nuclei and viewed by confocal microscopy on a Leica STED system.

Opsonophagocytic Killing Assay

Polymorphonuclear leukocytes (PMNs) were isolated from donor blood (collected in K3 EDTA tubes (Greiner Bio-One)) using Polymorphprep™ (Axis-Shield) as per manufacturer's instructions, and were resuspended in assay buffer (RPMI (Gibco) supplemented with 0.15 mM CaCl₂), 0.5 mM MgCl₂ and 0.5% (v/v) human serum albumin). N. gonorrhoeae 1291 (˜1×10³ CFU) was incubated in serial-dilutions of MAb 6E4 (0-10 μg) for 15 min at 37° C. PMNs (˜1×10⁵ cells) and a complement source (10% v/v normal human serum pre-absorbed with N. gonorrhoeae) were then added, and incubated at 37° C. for 90 min. N. gonorrhoeae survival was determined after plating of serial dilutions on GC agar, and survival calculated as a percentage relative to no-antibody control. Statistical significance was calculated using one-way analysis of variance (ANOVA) and Student's t-test.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

What is claimed is:

1. A chimeric molecule comprising, consisting or consisting essentially of a carrier and an oligosaccharide comprising an oligosaccharide sequence corresponding to a Neisseria lipooligosaccharide, wherein the oligosaccharide sequence comprises keto-deoxyoctulosonate (KDO) as a terminal saccharide unit.

2. The chimeric molecule of claim 1, wherein the oligosaccharide sequence comprises KDOα(2→3)Gal.

3. The chimeric molecule of claim 1, wherein the oligosaccharide sequence comprises KDOα(2→3)Galβ(1→4)GlcNAc.

4. The chimeric molecule of any one of claims 1 to 3, wherein the oligosaccharide comprises n occurrences of the oligosaccharide sequence, wherein n is 1 to 4.

5. The chimeric molecule of claim 4, wherein n is 2.

6. The chimeric molecule of claim 4 or claim 5, wherein respective oligosaccharide sequences are connected to one another by a linker.

7. The chimeric molecule of any one of claims 1 to 6, wherein the carrier is connected to the oligosaccharide by a linker.

8. The chimeric molecule of any one of claims 1 to 7, wherein the carrier is a protein, a peptide, a lipid, a polymer, a dendrimer, a virosome, a virus-like particle (VLP), an outer membrane vesicle or a combination thereof.

9. The chimeric molecule of any one of claims 1 to 8, wherein the carrier is protein carrier.

10. The chimeric molecule of claim 9, wherein the protein carrier is selected from a bacterial toxoid, a toxin, an exotoxin, and a nontoxic derivative thereof, such as keyhole limpet hemocyanine (KLH), hepatitis B virus core protein, thyroglobulin, albumins (such as bovine serum albumin (BSA), human serum albumin (HSA), and ovalbumin), pneumococcal surface protein A (PspA), pneumococcal adhesin protein (PsaA), purified protein derivative of tuberculin (PPD); transferrin binding proteins, polyamino acids, such as poly(lysine:glutamic acid), tetanus toxoid, tetanus toxin Fragment C, diphtheria toxoid, CRM (a nontoxic diphtheria toxin mutant), cholera toxoid, Staphylococcus aureus exotoxins or toxoids, Escherichia coli heat labile enterotoxin, Pseudomonas aeruginosa exotoxin A and bacterial outer membrane proteins (such as N. meningitidis serotype B outer membrane protein complex (OMPC) and outer membrane class 3 porin (rPorB)).

11. The chimeric molecule of claim 9, wherein the protein carrier is CRM197.

12. The chimeric molecule of any one of claims 1 to 11, wherein the a Neisseria lipooligosaccharide is a lipooligosaccharide of N. gonorrhoeae or N. meningitidis.

13. A composition comprising, consisting or consisting essentially of the chimeric molecule of any one of claims 1 to 12, and a pharmaceutically acceptable vehicle or adjuvant.

14. The composition of claim 13, further comprising an ancillary agent for treating or inhibiting the development of a Neisseria infection.

15. The composition of claim 14, wherein the ancillary agent is an antimicrobial agent.

16. The composition of claim 14, wherein the ancillary agent is an ancillary immunogen (e.g., proteinaceous immunogens derived from Neisseria antigens such as AniA, MsrAB, MetQ, NhbA, etc.) for eliciting an immune response to a Neisseria organism.

17. A method of eliciting an immune response to a Neisseria organism in a subject, the method comprising, consisting or consisting essentially of immunizing the subject with the chimeric molecule of any one of claims 1 to 12, or the composition of any one of claims 13 to 16, to thereby elicit an immune response to the Neisseria organism in the subject.

18. The method of claim 17, wherein the Neisseria organism is N. gonorrhoeae or N. meningitidis.

19. The method of claim 17 or claim 18, wherein the Neisseria organism is resistant to at least one antibacterial drug.

20. A method of producing an antigen-binding molecule that binds specifically with a Neisseria organism, the method comprising: (1) immunizing an animal with the chimeric molecule of any one of claims 1 to 12, or the composition of any one of claims 13 to 16; (2) detecting a B cell from the animal, which binds specifically with the chimeric molecule; and (3) isolating the antigen-binding molecule expressed by that B cell.

21. A method of producing an antigen-binding molecule that binds specifically with a Neisseria organism, the method comprising: (1) screening a library of antigen-binding molecules with the chimeric molecule of any one of claims 1 to 12, or the composition of any one of claims 13 to 16; (2) detecting an antigen-binding molecule that binds specifically with the chimeric molecule, or Neisseria organism or LOS thereof; and (3) isolating the detected antigen-binding molecule.

22. An antigen-binding molecule produced by the method of claim 20 or claim 21, or a derivative antigen-binding molecule with the same epitope-binding specificity as the antigen-binding molecule.

23. The antigen-binding molecule of claim 22, wherein the derivative antigen-binding molecule is selected from antibody fragments (such as Fab, Fab′, F(ab′)₂, Fv), single chain (scFv) and domain antibodies (including, for example, shark and camelid antibodies), and fusion proteins comprising an antibody, and any other modified configuration of the immunoglobulin molecule that comprises an antigen binding/recognition site.

24. A cell (e.g., a hybridoma or cell line) that produces the antigen-binding molecule of claim 22 or claim 23.

25. A method for treating or inhibiting the development of a Neisseria infection in a subject, the method comprising administering to the subject an effective amount of the chimeric molecule of any one of claims 1 to 12, or the composition of any one of claims 13 to 16, or the antigen-binding molecule of claim 22 or claim 23.

26. The method of claim 23, wherein the chimeric molecule or the antigen-binding molecule is administered concurrently with an ancillary agent (e.g., an antimicrobial agent and/or immunogen).

27. A kit for treating or inhibiting the development of a Neisseria infection in a subject, the kit comprising: the chimeric molecule of any one of claims 1 to 12, or the composition of any one of claims 13 to 16, or the antigen-binding molecule of claim 22 or claim 23, and optionally instructional material for performing the treatment or inhibition.
